SEQUENCE LISTING (36 sequences)

SEQ ID NO: 1 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 2 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 3 - DNA, 5436 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagggagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 4 - DNA, 5312 bp, Homo sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgatgcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgacaaaaaaaaaaaaaaa

SEQ ID NO: 5 - RNA, 25 nt, Homo sapiens
gacaguggaaguuuuuuuuuccucg

SEQ ID NO: 6 - RNA, 19 nt, Homo sapiens
auuagugucaucuugccuc

SEQ ID NO: 7 - RNA, 25 nt, Homo sapiens
aaugcccuacaucuuauuuuccuca

SEQ ID NO: 8 - RNA, 24 nt, Homo sapiens
gguucaagcgauucucgugccucg

SEQ ID NO: 9 - RNA, 24 nt, Homo sapiens
ggcugguccgaacuccugaccuca

SEQ ID NO: 10 - RNA, 21 nt, Homo sapiens
gauucacccaccuuggccuca

SEQ ID NO: 11 - RNA, 28 nt, Homo sapiens
ggguguuaagacuugacacaguaccucg

SEQ ID NO: 12 - RNA, 28 nt, Homo sapiens
agugcuuaugaggggauauuuaggccuc

SEQ ID NO: 13 - RNA, 21 nt, Homo sapiens
gaugcacccaccuuggccuca

SEQ ID NO: 14 - RNA, 22 nt, Homo sapiens
uugauauguuggaugauggagu

SEQ ID NO: 15 - RNA, 22 nt, Homo sapiens
uugguguguuggaugauggagu

SEQ ID NO: 16 - RNA, 22 nt, Homo sapiens
uugguauguuggaugauggagu

SEQ ID NO: 17 - RNA, 21 nt, Homo sapiens
ugauacguuggaugauggaga

SEQ ID NO: 18 - RNA, 20 nt, Homo sapiens
uauauguuggaggauggagu

SEQ ID NO: 19 - RNA, 22 nt, Homo sapiens
uugauauguuagaugauggagu

SEQ ID NO: 20 - RNA, 20 nt, Homo sapiens
gacauguuugaugauggagu

SEQ ID NO: 21 - RNA, 21 nt, Homo sapiens
ugucguguuuguugauggagu

SEQ ID NO: 22 - RNA, 24 nt, Homo sapiens
ugagguaguagguugugugcuuuu

SEQ ID NO: 23 - DNA, 21 bp, Homo sapiens
cctgacctcaagtgatgcacc

SEQ ID NO: 24 - DNA, 21 bp, Artificial Sequence
  recombinant
tgtgcatcacuugaggucagg

SEQ ID NO: 25 - RNA, 21 nt, Artificial Sequence
  recombinant
ugugcaucacuugaggucagg

SEQ ID NO: 26 - RNA, 21 nt, Artificial Sequence
  recombinant
ggugcaucacuugaggucagg

SEQ ID NO: 27 - DNA, 21 bp, Artificial Sequence
  recombinant
ugaccucaagugatgcaccca

SEQ ID NO: 28 - DNA, 21 bp, Homo sapiens
ctcctgacctcaagtgatgca

SEQ ID NO: 29 - RNA, 21 nt, Artificial Sequence
  recombinant
ugcaucacuugaggucaggag

SEQ ID NO: 30 - DNA, 21 bp, Artificial Sequence
  recombinant
tgcatcacuugaggucaggag

SEQ ID NO: 31 - RNA, 21 nt, Artificial Sequence
  recombinant
ccugaccucaagugaugcacc

SEQ ID NO: 32 - DNA, 21 bp, Artificial Sequence
  recombinant
ccugaccucaagugatgcacc

SEQ ID NO: 33 - DNA, 21 bp, Homo sapiens
actcctgacctcaagtgatgc

SEQ ID NO: 34 - RNA, 21 nt, Artificial Sequence
  recombinant
ucaucacuugaggucaggagu

SEQ ID NO: 35 - DNA, 21 bp, Artificial Sequence
  recombinant
uccugaccucaagtgatgcac

SEQ ID NO: 36 - RNA, 21 nt, Artificial Sequence
  recombinant
uccugaccucaagugaugcac

(19) United States
(12) Patent Application Publication
(10) Pub. No.: US 2011/0212021 A1
(43) Pub. Date: Sep. 1, 2011

(54) TARGETED OLIGONUCLEOTIDE COMPOSITIONS FOR MODIFYING GENE EXPRESSION

(76) Inventors: Frank J. Slack, (US); Joanne B. Weidhaas, (US)

(21) Appl. No.: 12/995,280

(22) PCT Filed: May 29, 2009
(86) PCT No.: PCT/US2009/045648

§ 371 Date: May 3, 2011

Related U.S. Application Data
(60) Provisional application No. 61/130,528, filed on May 30, 2008.

(51) Int. Cl. A61K 51/00; C12N 5/09; C07H 21/00; C07H 21/02; A61K 31/7088; A61P 35/00

(52) U.S. Cl. 424/1.11; 435/375; 536/24.5; 514/44 A

(57) ABSTRACT

The invention comprises compositions and methods for modifying gene expression. Modified oligos of the invention restore the lost function of let-7 wild type miRNA molecules that are prevented from silencing target genes by mutations occurring within their binding sites. Administration of a particular modified oligo (SEQ ID NO: 22) leads to increased cell death in cancer cells carrying the LCS6 SNP.

### RELATED APPLICATIONS

This application is related to provisional application U.S. Ser. No. 61/130,528, filed May 30, 2008, the contents which are herein incorporated by reference in their entirety.

### FIELD OF THE INVENTION

This invention relates generally to the fields of cancer and molecular biology. The invention provides compositions and methods for modifying the expression of genes that cause a variety of disorders, such as cancer.

### BACKGROUND OF THE INVENTION

Cancer is often caused by novel mutations that occur within regulatory sequences of genes controlling essential cellular functions such as DNA repair, cell cycle, proliferation, and cell adhesion. Current therapies are unable to address the underlying genetic mechanisms that lead to the development of the cancer. As such, there exists a long-felt need for a new kind of cancer therapy that is individualized and that addresses the underlying genetic and molecular mechanisms operating in cancer cells.

### SUMMARY OF THE INVENTION

The compositions and methods of the invention provide a new form of genetic and molecular therapy capable of treating cancer. Pharmaceutical compositions of the invention are individualized and optimized to target misregulated genes and/or specific mutations present in the genetic and epigenetic backgrounds of the intended subject. In one aspect of the invention, therapeutic compositions restore functions that are lost when novel or inherited mutations in miRNA binding sites modify wild type miRNA binding efficacies such that target genes are no longer sufficiently silenced in order to prevent the development of cancer.

The invention provides a composition containing the above isolated modified oligo. In certain embodiments, compositions including isolated modified oligos also include a pharmaceutically acceptable carrier. In other embodiments of the invention, this composition further comprises a cytotoxic compound. Exemplary cytotoxic compounds include, but are not limited to, all radioactive isotopes and chemotherapeutic compounds.

The invention provides a method of treating or alleviating a symptom of a cell proliferative disorder comprising administering to a subject in need thereof a composition of the invention. In a preferred embodiment, the cell proliferative disorder is cancer. Accordingly, the invention provides a method of treating or alleviating a symptom of cancer comprising administering to a subject in need thereof a composition of the invention. Exemplary cancers encompassed by the invention include, but are not limited to, all varieties of lung cancer, ovarian cancer, breast cancer, uterine cancer, head and neck cancer, pancreatic cancer, prostate cancer, renal cancer or colon cancer. In one aspect of the invention, the subject carries the LSC6 SNP.

Compositions are administered locally using the methods provided herein. Alternatively, or in addition, compositions are administered systemically using the methods provided herein. Compositions are administered intravenously, intradermally, subcutaneously, orally, transdermally, topically, transmucosally, transopthalmically, intratracheally, intranasally, epidermally, intraperitoneally, intraorbitally, intraarterially, intracapsularly, intraspinally, intrasternally, intracranially, intrathecally, intraventricularly, parenterally, non-parenterally, rectally, or surgically. Furthermore, multiple compositions may be administered to the same subject, either sequentially or simultaneously by any of the listed or art-recognized methods.

The invention provides methods of inducing cell death in a cancer cell comprising contacting a modified oligo composition to the cancer cell. The methods herein encompass cancer cells that are in vivo, in vitro, ex vivo, and in situ. Cancer cells of the methods include cells from all varieties of cancer including, but not limited to, lung cancer, ovarian cancer, breast cancer, uterine cancer, head and neck cancer, pancreatic cancer, prostate cancer, renal cancer, or colon cancer. In one aspect of the invention, the cancer cell contacted using the methods herein contains a mutation. In a preferred embodiment, the mutation is the LCS6 SNP. Alternatively, the cancer cell contains the LCS6 SNP.

Compositions of the invention are contacted to a cancer cell using the instant methods by local or systemic administration. Compositions contact a cancer cell following, for example, topical, intravenous, intraocular, subcutaneous, intraparitoneal, intramuscular, intraspinal, or surgical administration.

### BRIEF DESCRIPTION OF THE DRAWINGS

FIG. 1A is the sequence of the modified oligo (SEQ ID NO: 22) used in FIG. 1B.

FIG. 1B is a graph showing the proportional survival of cancer cells, wild type (left two columns) or mutant (containing the LCS6 SNP mutation, right two columns) following exposure to wild type (prelet-7b), modified let-7b oligo, or antisense let-7 (anti-let-7) miRNAs.

FIG. 2A is a table showing the values of normalized luciferase expression and the standard deviations of the mean for each of three modified oligos when luciferase reporter proteins are fused to either wild type and LCS6 SNP containing KRAS 3′UTR regions. Values are normalized to a control miRNA construct such that the expression of the control is equal to 1.

FIG. 2B is a graphic representation of the values presented in FIG. 2A.

FIG. 3 is a graphic representation of a clonogenic assay demonstrating the ability of different combinations of oligonucleotides (1-2, 1-3, 2-1, 2-3, and 3-2) to silence KRAS expression, and therefore, affect cell survival of oncogenic cell lines containing the LCS6 SNP (also referred to as the onco-SNP).

FIG. 4 is a graphic representation of a clonogenic assay demonstrating the ability of different combinations of oligonucleotides (1.2, 1.3, 2.1, and 2.3) to silence KRAS expression, and therefore, affect cell survival of ovarian and pancreatic cancer cell lines containing the LCS6 SNP. Cell survival was normalized to ovarian and pancreatic cancer cell lines that do not contain the LCS6 SNP and expressed as a percentage of the survival observed in the non-SNP cell lines.

FIG. 5 is a graphic representation of a clonogenic assay demonstrating the ability of different combinations of oligonucleotides (1.2, 1.3, and 2.1) to silence KRAS expression, and therefore, affect cell survival of an ovarian cancer cell line containing the LCS6 SNP. Cell survival was normalized to an ovarian cancer cell line that does not contain the LCS6 SNP.

### DETAILED DESCRIPTION

The invention is based upon the unexpected discovery that modified or synthetic oligonucleotide (referred to herein as “oligo”) molecules restore or improve gene silencing mechanisms or functions that are compromised or lost in cells that cause a variety of disorders, such as cancer. Specifically, modified or synthetic oligo molecules of the invention are administered to silence the overexpression of oncogenes. The invention is based in part on mutations present within the binding sites, or complementary sites, of wild type miRNAs which modify the binding efficacy of these miRNAs and affect gene silencing. When mutations occur within miRNA binding sites present within proto-oncogenes or oncogenes, the gene silencing functions of at least one wild type miRNA are affected resulting in the overexpression of this proto-oncogene or oncogene. Modified or synthetic oligos of the invention are engineered to bind to miRNA binding sites such that gene silencing is either restored, in the case of a mutation which modifies wild type miRNA binding, or enhanced, in the event that a gene is misregulated and overexpressed in the absence of a mutation.

In one aspect of the invention, modified or synthetic oligos are engineered to bind to a let-7 complementary site (LCS) within the KRAS gene or transcript that contains the LCS6 SNP. Subjects who carry the LCS6 SNP display altered expression of this gene compared to subjects who do not carry this mutation. In many subjects who carry the LCS6 SNP, KRAS is overexpressed. KRAS contains eight let-7 complementary sites (LCSs), however, a single nucleotide polymorphism (SNP) occurring at the 4th position of LCS6 (SEQ ID NO: 21) is predictive of the occurrence of cancer. The presence of the LCS6 SNP modifies the binding efficacy of wild type let-7 miRNAs. As a result of the LCS6 SNP, KRAS expression is not sufficiently silenced by wild type let-7 miRNAs. The consequence of carrying the LCS6 SNP is the development of cancer.

Accordingly, the invention provides compositions and methods for modifying gene expression in subjects who carry mutations in let-7 complementary sites in order to treat, or alleviate a sign or symptom of, cancer. The invention also encompasses subjects who are not carriers of such mutations, because genes regulated by let-7 are up-regulated in many cancer types. The compositions and methods provided herein also provide a therapeutic benefit to these subjects.

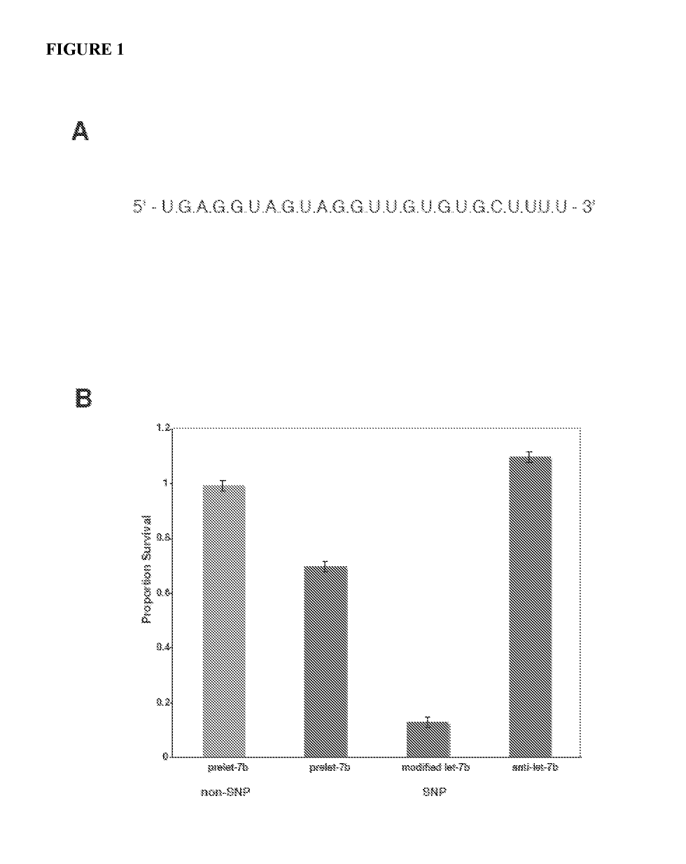

For example, using the compositions and methods provided herein, a modified oligo (SEQ ID NO: 22) that has the ability to base-pair with the LCS6 SNP in the KRAS 3′UTR is delivered to human cancer cells that contain the LCS6 SNP. It was demonstrated that this modified oligo (SEQ ID NO: 22) dramatically reduced cell survival in these cells, more so than a naturally occurring miRNA precursor, let-7, that is also predicted to target this site (FIG. 1). These results demonstrate the unique ability of using these targeting oligonucleotides to reduce growth and/or stimulate death of cancer cells containing the LCS6 SNP. Moreover, the data support using this approach as a therapy in all cancers containing the LCS6 SNP.

### Single Nucleotide Polymorphisms (SNPs)

A single nucleotide polymorphism (SNP) is a DNA sequence variation occurring when a single nucleotide in the genome (or other shared sequence) differs between members of a species (or between paired chromosomes in an individual). SNPs may fall within coding sequences of genes, non-coding regions of genes, or in the intergenic regions between genes. SNPs within a coding sequence will not necessarily change the amino acid sequence of the protein that is produced, due to degeneracy of the genetic code. A SNP mutation that results in a new DNA sequence that encodes the same polypeptide sequence is termed synonymous (also referred to as a silent mutation). Conversely, a SNP mutation that results in a new DNA sequence that encodes a different polypeptide sequence is termed non-synonymous. SNPs that are not in protein-coding regions may still have consequences for gene splicing, transcription factor binding, or the sequence of non-coding RNA.

SNPs occurring within non-coding regions of genes, e.g. untranslated regions, are particularly important because those regions contain regulatory sequences which are complementary to miRNA molecules and required for interaction with other regulatory factors. SNPs occurring within genomic sequences are transcribed into mRNA transcripts which are targeted by miRNA molecules for degradation or translational silencing. SNPs occurring within the 3′ untranslated region (UTR) of the genomic sequence or mRNA transcript of a gene are of particular importance to the methods of the invention.

### MicroRNAs

MicroRNAs (miRNAs) are small, non-coding RNAs, recently identified genetic regulators that control cell metabolism, development, cell cycle, cell differentiation and cell death. In addition, miRNAs have been found to be important in cancer, aging, and other disease states, likely due to their ability to regulate hundreds of genes targets.

mRNAs act by inhibiting translation of messenger RNA (mRNA) into protein by binding to the 3′ untranslated region (UTR) of their target mRNAs. It has been found that these microRNA binding sites in 3′UTRs are very highly conserved regions in humans, suggesting an important role in these regions in natural selection. The high degree of conservation of the 3′UTR supports the hypothesis that a disruption of this region leads to disease. While not bound by theory, miRNAs inhibit mRNA translation by either causing mRNA degradation or inhibiting translation itself.

MiRNAs are single-stranded RNA molecules of about 21-23 nucleotides in length. MiRNAs are encoded by endogenous and exogenous genes that are transcribed from DNA by RNA polymerase II, however, miRNA are never translated into polypeptide sequences. As such, miRNA are considered in the art as “non-coding RNA.” The term “endogenous” gene as used herein is meant to encompass all genes that naturally occur within the genome of an individual. The term “exogenous” gene as used herein is meant to encompass all genes that do not naturally occur within the genome of an individual. For example, a miRNA could be introduced exogenously by a virus.

While not limited by theory, the present invention includes and is based in part on the understanding that miRNA biogenesis occurs by the following mechanism. MiRNA are processed from primary mRNA transcripts, called “pri-miRNA” by the nuclease Drosha and the double-stranded RNA binding protein DGCR8/Pasha. Once processed, these transcripts form stem-loop structures referred to as “pre-miRNA”. Pre-miRNA are processed one step further by the endonuclease Dicer, which transforms the double-stranded pre-miRNA molecules into the single-stranded mature miRNA and initiates formation of the RNA-induced silencing complex (RISC). One of the two resulting single-stranded complementary miRNA strands, the guide strand, is selected by the argonaute protein of the RISC and incorporated into the RISC, while the other strand, the anti-guide or passenger strand, is degraded. Following integration into the RISC, miRNAs bind target mRNAs and subsequently inhibit translation.

mRNAs are complementary to a part of one or more mRNAs. Moreover, miRNAs do not require absolute sequence complementarity to bind an mRNA, enabling them to regulate a wide range of target transcripts. As used herein the term “absolute sequence complementarity” is meant to describe a requirement that each nucleotide pair along the length of two sequences, e.g. a miRNA and a target gene or transcript, bind without gaps. It is common that miRNAs bind to their complementary sites with a lesser degree of complementarity. MiRNAs typically bind target sequences with gaps between matched nucleotides. As used herein, the term “complementary” is meant to describe two sequences in which at least 50% of the nucleotides bind from one sequence to the other sequence in trans.

mRNAs have a seed sequence that consists of nucleotides 2-7 or 2-8 at the 5′ end of the sequence. The seed sequence binds more closely follows the traditional rules of Watson-Crick base pairing that the 3′ sequence of the miRNA. Sequence complementarity between the seed sequence of the miRNA and its target is both necessary and sufficient to determine miRNA binding. Both experimental and computational approaches have determined that complementarity of seven or more base pairs at the miRNA 5′ end are sufficient to confer regulation in vivo and are used in biologically relevant targets (Brenneke et al. PLoS Biology. 2005, 3(3): e85). Brenneke et al. also found that extensive complementarity to the 3′ sequence of the miRNA is not sufficient to confer regulation without a minimal element of 5′ complementarity within the seed sequence. However, the complementarity of the miRNA 3′ sequence to its target may determine specificity of miRNAs within a family. Within the seed sequence, G:U base-pairs, single-nucleotide bulges, and mismatches occur which compromise the ability of the miRNA to bind its target. If the seed sequence binding is weakened due to these irregularities, greater complementarity is required within the 3′ end of the miRNA to enable efficacious binding of the miRNA to its target. Thus, in order to predict miRNA binding, miRNA sequences and their target sites can be categorized into two groups: those miRNAs for which perfect base pairing occurs at the 5′ end and those miRNA for which seed sequence pairing is weaker and the 3′ sequence compensates with stronger complementarity. Brenneke et al. refer to these groups as 5′ dominant and 3′ compensatory, respectively. Exemplary 3′ compensatory sites include, but are not limited to, smaller seed matches of 4-6 base-pairs and those seed matches with 7-8 bases and interruptions such as G:U bases, single nucleotide bulges, or mismatches, each of which are accompanied by stronger 3′ complementarity.

Modified oligonucleotides of the invention are 5′ dominant or 3′ compensatory. Moreover, in certain circumstances, modified oligonucleotides of the invention bind to “canonical” miRNA sites, in which the seed sequence and the 3′ sequence of the miRNA both bind strongly to the target sequence. Alternatively, modified oligonucleotides of the invention bind to “seed sites” in which the seed sequence of the miRNA alone binds strongly to the target sequence and mediates silencing irrespective of the binding complementarity of the 3′ sequence of the miRNA to the target sequence.

mRNAs are frequently complementary to the 3′ UTR of the mRNA transcript. Alternatively, or in addition, miRNAs target methylation genomic sites which correspond to genes encoding targeted mRNAs. The methylation state of genomic DNA in part determines the accessibility of that DNA to transcription factors. As such, DNA methylation and de-methylation regulate gene silencing and expression, respectively.

### Oncogenic and Tumor Suppressor MiRNAs

MiRNAs that silence expression of tumor suppressor genes are oncogenes. Alternatively, miRNAs are tumor suppressor genes, which silence the translation of mRNAs transcripts of oncogenes. The term “oncogene” as used herein is meant to encompass any gene that, when expressed, directly or indirectly, causes a cell to enter the cell cycle at an inappropriate time or by an uncontrolled mechanism, or fail to die appropriately. Exemplary oncogenes include, but are not limited to, growth factors, transcription factors, regulatory proteins, e.g. GTPases and receptors, and cell cycle proteins. The term “proto-oncogene” as used herein is meant to encompass any gene, which if modified, directly or indirectly, causes a cell to inappropriately enter the cell cycle. Examples of proto-oncogenes include, but are not limited to, RAS, WNT, MYC, ERK and TRK. The term “tumor suppressor gene” as used herein encompasses any gene that repressed or silenced, leads deregulated cell division and/or overexpression of a proto-oncogene or oncogene. Exemplary tumor suppressor genes include, but are not limited to, retinoblastoma (encoding the Rb protein), TP53 (encoding the p53 protein), PTEN, APC, and CD95. Tumor suppressor gene products repress genes that are essential for the continuing of the cell cycle. Effectively, if these genes are not expressed, the cell cycle will not continue, effectively inhibiting cell division. Tumor suppressor gene products couple the cell cycle to DNA damage. Thus, these gene products activate cell cycle checkpoints and DNA repair mechanisms that stall or prevent cell division. If the damage cannot be repaired, the cell initiate apoptosis, or programmed cell death. Some tumor suppressor gene products are involved in cell adhesion, and thus, prevent tumor cells from dispersing, block loss of contact inhibition, and inhibit metastasis. These proteins are also known as metastasis suppressors.

SNPs within the binding site of a tumor suppressing miRNA that decrease binding efficacy, and therefore oncogene silencing, lead to an increased risk, susceptibility or probability of presenting one or more symptoms of a cell proliferative disease. Similarly, SNPs within the binding site of an oncogenic miRNA that increase binding, and therefore increase gene repression, lead to an increased risk, susceptibility or probability of presenting one or more symptoms of a cell proliferative disease.

The invention includes all known tumor suppressor and oncogenic miRNAs and their corresponding complementary binding sites. Moreover, all endogenous human miRNAs are encompassed by the invention, the names, sequences, and targets of which are provided at the database of the Wellcome Trust Sanger Institute MicroRNA Listing for Homo sapiens, the entirety of which is herein incorporated by reference.

### KRAS Gene

The KRAS gene is one form of RAS in humans. The RAS gene encodes for a protein belongs to a larger superfamily of small GTPases that include the Ras, Rho, Arf, Rab, and Ran families. Functionally, GTPase proteins are molecular switches for a wide variety of signal transduction pathways that control practically every function within a cell. Exemplary functions regulated by GTPase proteins are cytoskeletal integrity, cell proliferation, cell adhesion, apoptosis, and cell migration. Thus, Ras protein deregulated within a cell often leads to increased cell invasion, metastasis, and decreased apoptosis. Importantly, Ras protein is attached to the cell membrane by prenylation and couples growth factor receptors to downstream mitogenic effectors involved in cell proliferation or differentiation.

There are three human RAS genes comprising HRAS, KRAS, and NRAS. Each gene comprises multiple miRNA complementary sites in the 3′UTR of their mRNA transcripts. Specifically, each human RAS gene comprises multiple let-7 complementary sites (LCSs).

Importantly, KRAS is capable of acting as a tumor suppressor gene, a protooncogene, or an oncogene. SNPs in the 3′UTR of KRAS may lead to either increased or decreased binding efficacy of wild type miRNAs. The SNP which occurs in LCS6 (shown below in SEQ ID NOs: 3 and 4), modifies the binding of let-7 family miRNAs. In one aspect of the invention, KRAS acts as a proto-oncogene or oncogene, the LCS6 SNP decreases the binding efficacy of at least one miRNA, causing expressing of the oncogene to be augmented, and the LCS6 SNP is a marker of cell proliferative disease. In another aspect of the invention, KRAS acts as a tumor suppressor gene, the LCS6 SNP increases the binding efficacy of at least one miRNA, causing expression of the tumor suppressor gene to be repressed, and the LCS6 SNP is a marker of cell proliferative disease.

Human KRAS, transcript variant a, is encoded by the following mRNA sequence (NCBI Accession No. NM_{—}033360 and SEQ ID NO: 1) (untranslated regions are bolded, LCS6 is underlined, all sequences provided herein are given from 5′ to 3′):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gagagtggag gatgcttttt atacattggt |
| 661 | gagggagatc cgacaataca gattgaaaaa aatcagcaaa gaagaaaaga ctcctggctg |
| 721 | tgtgaaaatt aaaaaatgca ttataatgta atctgggtgt tgatgatgcc ttctatacat |
| 781 | tagttcgaga aattcgaaaa cataaagaaa agatgagcaa agatggtaaa aagaagaaaa |
| 841 | agaagtcaaa gacaaagtgt gtaattatgt aaatacaatt tgtacttttt tcttaaggca |
| 901 | tactagtaca agtggtaatt tttgtacatt acactaaatt attagcattt gttttagcat |
| 961 | tacctaattt ttttcctgct ccatgcagac tgttagcttt taccttaaat gcttatttta |
| 1021 | aaatgacagt ggaagttttt ttttcctcta agtgccagta ttcccagagt tttggttttt |
| 1081 | gaactagcaa tgcctgtgaa aaagaaactg aatacctaag atttctgtct tggggttttt |
| 1141 | ggtgcatgca gttgattact tcttattttt cttaccaatt gtgaatgttg gtgtgaaaca |
| 1201 | aattaatgaa gcttttgaat catccctatt ctgtgtttta tctagtcaca taaatggatt |
| 1261 | aattactaat ttcagttgag accttctaat tggtttttac tgaaacattg agggaacaca |
| 1321 | aatttatggg cttcctgatg atgattcttc taggcatcat gtcctatagt ttgtcatccc |
| 1381 | tgatgaatgt aaagttacac tgttcacaaa ggttttgtct cctttccact gctattagtc |
| 1441 | atggtcactc tccccaaaat attatatttt ttctataaaa agaaaaaaat ggaaaaaaat |
| 1501 | tacaaggcaa tggaaactat tataaggcca tttccttttc acattagata aattactata |
| 1561 | aagactccta atagcttttc ctgttaaggc agacccagta tgaaatgggg attattatag |
| 1621 | caaccatttt ggggctatat ttacatgcta ctaaattttt ataataattg aaaagatttt |
| 1681 | aacaagtata aaaaattctc ataggaatta aatgtagtct ccctgtgtca gactgctctt |
| 1741 | tcatagtata actttaaatc ttttcttcaa cttgagtctt tgaagatagt tttaattctg |
| 1801 | cttgtgacat taaaagatta tttgggccag ttatagctta ttaggtgttg aagagaccaa |
| 1861 | ggttgcaagg ccaggccctg tgtgaacctt tgagctttca tagagagttt cacagcatgg |
| 1921 | actgtgtccc cacggtcatc cagtgttgtc atgcattggt tagtcaaaat ggggagggac |
| 1981 | tagggcagtt tggatagctc aacaagatac aatctcactc tgtggtggtc ctgctgacaa |
| 2041 | atcaagagca ttgcttttgt ttcttaagaa aacaaactct tttttaaaaa ttacttttaa |
| 2101 | atattaactc aaaagttgag attttggggt ggtggtgtgc caagacatta attttttttt |
| 2161 | taaacaatga agtgaaaaag ttttacaatc tctaggtttg gctagttctc ttaacactgg |
| 2221 | ttaaattaac attgcataaa cacttttcaa gtctgatcca tatttaataa tgctttaaaa |
| 2281 | taaaaataaa aacaatcctt ttgataaatt taaaatgtta cttattttaa aataaatgaa |
| 2341 | gtgagatggc atggtgaggt gaaagtatca ctggactagg aagaaggtga cttaggttct |
| 2401 | agataggtgt cttttaggac tctgattttg aggacatcac ttactatcca tttcttcatg |
| 2461 | ttaaaagaag tcatctcaaa ctcttagttt ttttttttta caactatgta atttatattc |
| 2521 | catttacata aggatacact tatttgtcaa gctcagcaca atctgtaaat ttttaaccta |
| 2581 | tgttacacca tcttcagtgc cagtcttggg caaaattgtg caagaggtga agtttatatt |
| 2641 | tgaatatcca ttctcgtttt aggactcttc ttccatatta gtgtcatctt gcctccctac |
| 2701 | cttccacatg ccccatgact tgatgcagtt ttaatacttg taattcccct aaccataaga |
| 2761 | tttactgctg ctgtggatat ctccatgaag ttttcccact gagtcacatc agaaatgccc |
| 2821 | tacatcttat ttcctcaggg ctcaagagaa tctgacagat accataaagg gatttgacct |
| 2881 | aatcactaat tttcaggtgg tggctgatgc tttgaacatc tctttgctgc ccaatccatt |
| 2941 | agcgacagta ggatttttca aacctggtat gaatagacag aaccctatcc agtggaagga |
| 3001 | gaatttaata aagatagtgc tgaaagaatt ccttaggtaa tctataacta ggactactcc |
| 3061 | tggtaacagt aatacattcc attgttttag taaccagaaa tcttcatgca atgaaaaata |
| 3121 | ctttaattca tgaagcttac tttttttttt tggtgtcaga gtctcgctct tgtcacccag |
| 3181 | gctggaatgc agtggcgcca tctcagctca ctgcaacctc catctcccag gttcaagcga |
| 3241 | ttctcgtgcc tcggcctcct gagtagctgg gattacaggc gtgtgccact acactcaact |
| 3301 | aatttttgta tttttaggag agacggggtt tcaccctgtt ggccaggctg gtctcgaact |
| 3361 | cctgacctca agtgattcac ccaccttggc ctcataaacc tgttttgcag aactcattta |
| 3421 | ttcagcaaat atttattgag tgcctaccag atgccagtca ccgcacaagg cactgggtat |
| 3481 | atggtatccc caaacaagag acataatccc ggtccttagg tagtgctagt gtggtctgta |
| 3541 | atatcttact aaggcctttg gtatacgacc cagagataac acgatgcgta ttttagtttt |
| 3601 | gcaaagaagg ggtttggtct ctgtgccagc tctataattg ttttgctacg attccactga |
| 3661 | aactcttcga tcaagctact ttatgtaaat cacttcattg ttttaaagga ataaacttga |
| 3721 | ttatattgtt tttttatttg gcataactgt gattctttta ggacaattac tgtacacatt |
| 3781 | aaggtgtatg tcagatattc atattgaccc aaatgtgtaa tattccagtt ttctctgcat |
| 3841 | aagtaattaa aatatactta aaaattaata gttttatctg ggtacaaata aacaggtgcc |
| 3901 | tgaactagtt cacagacaag gaaacttcta tgtaaaaatc actatgattt ctgaattgct |
| 3961 | atgtgaaact acagatcttt ggaacactgt ttaggtaggg tgttaagact tacacagtac |
| 4021 | ctcgtttcta cacagagaaa gaaatggcca tacttcagga actgcagtgc ttatgagggg |
| 4081 | atatttaggc ctcttgaatt tttgatgtag atgggcattt ttttaaggta gtggttaatt |
| 4141 | acctttatgt gaactttgaa tggtttaaca aaagatttgt ttttgtagag attttaaagg |
| 4201 | gggagaattc tagaaataaa tgttacctaa ttattacagc cttaaagaca aaaatccttg |
| 4261 | ttgaagtttt tttaaaaaaa gctaaattac atagacttag gcattaacat gtttgtggaa |
| 4321 | gaatatagca gacgtatatt gtatcatttg agtgaatgtt cccaagtagg cattctaggc |
| 4381 | tctatttaac tgagtcacac tgcataggaa tttagaacct aacttttata ggttatcaaa |
| 4441 | actgttgtca ccattgcaca attttgtcct aatatataca tagaaacttt gtggggcatg |
| 4501 | ttaagttaca gtttgcacaa gttcatctca tttgtattcc attgattttt tttttcttct |
| 4561 | aaacattttt tcttcaaaca gtatataact ttttttaggg gatttttttt tagacagcaa |
| 4621 | aaactatctg aagatttcca tttgtcaaaa agtaatgatt tcttgataat tgtgtagtaa |
| 4681 | tgttttttag aacccagcag ttaccttaaa gctgaattta tatttagtaa cttctgtgtt |
| 4741 | aatactggat agcatgaatt ctgcattgag aaactgaata gctgtcataa aatgaaactt |
| 4801 | tctttctaaa gaaagatact cacatgagtt cttgaagaat agtcataact agattaagat |
| 4861 | ctgtgtttta gtttaatagt ttgaagtgcc tgtttgggat aatgataggt aatttagatg |
| 4921 | aatttagggg aaaaaaaagt tatctgcaga tatgttgagg gcccatctct ccccccacac |
| 4981 | ccccacagag ctaactgggt tacagtgttt tatccgaaag tttccaattc cactgtcttg |
| 5041 | tgttttcatg ttgaaaatac ttttgcattt ttcctttgag tgccaatttc ttactagtac |
| 5101 | tatttcttaa tgtaacatgt ttacctggaa tgtattttaa ctatttttgt atagtgtaaa |
| 5161 | ctgaaacatg cacattttgt acattgtgct ttcttttgtg ggacatatgc agtgtgatcc |
| 5221 | agttgttttc catcatttgg ttgcgctgac ctaggaatgt tggtcatatc aaacattaaa |
| 5281 | aatgaccact cttttaattg aaattaactt ttaaatgttt ataggagtat gtgctgtgaa |
| 5341 | gtgatctaaa atttgtaata tttttgtcat gaactgtact actcctaatt attgtaatgt |
| 5401 | aataaaaata gttacagtga caaaaaaaaa aaaaaa |

Human KRAS, transcript variant b, is encoded by the following mRNA sequence (NCBI Accession No. NM 004985 and SEQ ID NO: 2) (untranslated regions are bolded, LCS6 is underlined):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gggtgttgat gatgccttct atacattagt |
| 661 | tcgagaaatt cgaaaacata aagaaaagat gagcaaagat ggtaaaaaga agaaaaagaa |
| 721 | gtcaaagaca aagtgtgtaa ttatgtaaat acaatttgta cttttttctt aaggcatact |
| 781 | agtacaagtg gtaatttttg tacattacac taaattatta gcatttgttt tagcattacc |
| 841 | taattttttt cctgctccat gcagactgtt agcttttacc ttaaatgctt attttaaaat |
| 901 | gacagtggaa gttttttttt cctctaagtg ccagtattcc cagagttttg gtttttgaac |
| 961 | tagcaatgcc tgtgaaaaag aaactgaata cctaagattt ctgtcttggg gtttttggtg |
| 1021 | catgcagttg attacttctt atttttctta ccaattgtga atgttggtgt gaaacaaatt |
| 1081 | aatgaagctt ttgaatcatc cctattctgt gttttatcta gtcacataaa tggattaatt |
| 1141 | actaatttca gttgagacct tctaattggt ttttactgaa acattgaggg aacacaaatt |
| 1201 | tatgggcttc ctgatgatga ttcttctagg catcatgtcc tatagtttgt catccctgat |
| 1261 | gaatgtaaag ttacactgtt cacaaaggtt ttgtctcctt tccactgcta ttagtcatgg |
| 1321 | tcactctccc caaaatatta tattttttct ataaaaagaa aaaaatggaa aaaaattaca |
| 1381 | aggcaatgga aactattata aggccatttc cttttcacat tagataaatt actataaaga |
| 1441 | ctcctaatag cttttcctgt taaggcagac ccagtatgaa atggggatta ttatagcaac |
| 1501 | cattttgggg ctatatttac atgctactaa atttttataa taattgaaaa gattttaaca |
| 1561 | agtataaaaa attctcatag gaattaaatg tagtctccct gtgtcagact gctctttcat |
| 1621 | agtataactt taaatctttt cttcaacttg agtctttgaa gatagtttta attctgcttg |
| 1681 | tgacattaaa agattatttg ggccagttat agcttattag gtgttgaaga gaccaaggtt |
| 1741 | gcaaggccag gccctgtgtg aacctttgag ctttcataga gagtttcaca gcatggactg |
| 1801 | tgtccccacg gtcatccagt gttgtcatgc attggttagt caaaatgggg agggactagg |
| 1861 | gcagtttgga tagctcaaca agatacaatc tcactctgtg gtggtcctgc tgacaaatca |
| 1921 | agagcattgc ttttgtttct taagaaaaca aactcttttt taaaaattac ttttaaatat |
| 1981 | taactcaaaa gttgagattt tggggtggtg gtgtgccaag acattaattt tttttttaaa |
| 2041 | caatgaagtg aaaaagtttt acaatctcta ggtttggcta gttctcttaa cactggttaa |
| 2101 | attaacattg cataaacact tttcaagtct gatccatatt taataatgct ttaaaataaa |
| 2161 | aataaaaaca atccttttga taaatttaaa atgttactta ttttaaaata aatgaagtga |
| 2221 | gatggcatgg tgaggtgaaa gtatcactgg actaggaaga aggtgactta ggttctagat |
| 2281 | aggtgtcttt taggactctg attttgagga catcacttac tatccatttc ttcatgttaa |
| 2341 | aagaagtcat ctcaaactct tagttttttt tttttacaac tatgtaattt atattccatt |
| 2401 | tacataagga tacacttatt tgtcaagctc agcacaatct gtaaattttt aacctatgtt |
| 2461 | acaccatctt cagtgccagt cttgggcaaa attgtgcaag aggtgaagtt tatatttgaa |
| 2521 | tatccattct cgttttagga ctcttcttcc atattagtgt catcttgcct ccctaccttc |
| 2581 | cacatgcccc atgacttgat gcagttttaa tacttgtaat tcccctaacc ataagattta |
| 2641 | ctgctgctgt ggatatctcc atgaagtttt cccactgagt cacatcagaa atgccctaca |
| 2701 | tcttatttcc tcagggctca agagaatctg acagatacca taaagggatt tgacctaatc |
| 2761 | actaattttc aggtggtggc tgatgctttg aacatctctt tgctgcccaa tccattagcg |
| 2821 | acagtaggat ttttcaaacc tggtatgaat agacagaacc ctatccagtg gaaggagaat |
| 2881 | ttaataaaga tagtgctgaa agaattcctt aggtaatcta taactaggac tactcctggt |
| 2941 | aacagtaata cattccattg ttttagtaac cagaaatctt catgcaatga aaaatacttt |
| 3001 | aattcatgaa gcttactttt tttttttggt gtcagagtct cgctcttgtc acccaggctg |
| 3061 | gaatgcagtg gcgccatctc agctcactgc aacctccatc tcccaggttc aagcgattct |
| 3121 | cgtgcctcgg cctcctgagt agctgggatt acaggcgtgt gccactacac tcaactaatt |
| 3181 | tttgtatttt taggagagac ggggtttcac cctgttggcc aggctggtct cgaactcctg |
| 3241 | acctcaagtg attcacccac cttggcctca taaacctgtt ttgcagaact catttattca |
| 3301 | gcaaatattt attgagtgcc taccagatgc cagtcaccgc acaaggcact gggtatatgg |
| 3361 | tatccccaaa caagagacat aatcccggtc cttaggtagt gctagtgtgg tctgtaatat |
| 3421 | cttactaagg cctttggtat acgacccaga gataacacga tgcgtatttt agttttgcaa |
| 3481 | agaaggggtt tggtctctgt gccagctcta taattgtttt gctacgattc cactgaaact |
| 3541 | cttcgatcaa gctactttat gtaaatcact tcattgtttt aaaggaataa acttgattat |
| 3601 | attgtttttt tatttggcat aactgtgatt cttttaggac aattactgta cacattaagg |
| 3661 | tgtatgtcag atattcatat tgacccaaat gtgtaatatt ccagttttct ctgcataagt |
| 3721 | aattaaaata tacttaaaaa ttaatagttt tatctgggta caaataaaca ggtgcctgaa |
| 3781 | ctagttcaca gacaaggaaa cttctatgta aaaatcacta tgatttctga attgctatgt |
| 3841 | gaaactacag atctttggaa cactgtttag gtagggtgtt aagacttaca cagtacctcg |
| 3901 | tttctacaca gagaaagaaa tggccatact tcaggaactg cagtgcttat gaggggatat |
| 3961 | ttaggcctct tgaatttttg atgtagatgg gcattttttt aaggtagtgg ttaattacct |
| 4021 | ttatgtgaac tttgaatggt ttaacaaaag atttgttttt gtagagattt taaaggggga |
| 4081 | gaattctaga aataaatgtt acctaattat tacagcctta aagacaaaaa tccttgttga |
| 4141 | agttttttta aaaaaagcta aattacatag acttaggcat taacatgttt gtggaagaat |
| 4201 | atagcagacg tatattgtat catttgagtg aatgttccca agtaggcatt ctaggctcta |
| 4261 | tttaactgag tcacactgca taggaattta gaacctaact tttataggtt atcaaaactg |
| 4321 | ttgtcaccat tgcacaattt tgtcctaata tatacataga aactttgtgg ggcatgttaa |
| 4381 | gttacagttt gcacaagttc atctcatttg tattccattg attttttttt tcttctaaac |
| 4441 | attttttctt caaacagtat ataacttttt ttaggggatt tttttttaga cagcaaaaac |
| 4501 | tatctgaaga tttccatttg tcaaaaagta atgatttctt gataattgtg tagtaatgtt |
| 4561 | ttttagaacc cagcagttac cttaaagctg aatttatatt tagtaacttc tgtgttaata |
| 4621 | ctggatagca tgaattctgc attgagaaac tgaatagctg tcataaaatg aaactttctt |
| 4681 | tctaaagaaa gatactcaca tgagttcttg aagaatagtc ataactagat taagatctgt |
| 4741 | gttttagttt aatagtttga agtgcctgtt tgggataatg ataggtaatt tagatgaatt |
| 4801 | taggggaaaa aaaagttatc tgcagatatg ttgagggccc atctctcccc ccacaccccc |
| 4861 | acagagctaa ctgggttaca gtgttttatc cgaaagtttc caattccact gtcttgtgtt |
| 4921 | ttcatgttga aaatactttt gcatttttcc tttgagtgcc aatttcttac tagtactatt |
| 4981 | tcttaatgta acatgtttac ctggaatgta ttttaactat ttttgtatag tgtaaactga |
| 5041 | aacatgcaca ttttgtacat tgtgctttct tttgtgggac atatgcagtg tgatccagtt |
| 5101 | gttttccatc atttggttgc gctgacctag gaatgttggt catatcaaac attaaaaatg |
| 5161 | accactcttt taattgaaat taacttttaa atgtttatag gagtatgtgc tgtgaagtga |
| 5221 | tctaaaattt gtaatatttt tgtcatgaac tgtactactc ctaattattg taatgtaata |
| 5281 | aaaatagtta cagtgacaaa aaaaaaaaaa aa |

Human KRAS, transcript variant a, comprising the LCS6 SNP, is encoded by the following mRNA sequence (SEQ ID NO: 3) (untranslated regions are bolded, LCS6 is underlined, SNP is capitalized):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gagagtggag gatgcttttt atacattggt |
| 661 | gagggagatc cgacaataca gattgaaaaa aatcagcaaa gaagaaaaga ctcctggctg |
| 721 | tgtgaaaatt aaaaaatgca ttataatgta atctgggtgt tgatgatgcc ttctatacat |
| 781 | tagttcgaga aattcgaaaa cataaagaaa agatgagcaa agatggtaaa aagaagaaaa |
| 841 | agaagtcaaa gacaaagtgt gtaattatgt aaatacaatt tgtacttttt tcttaaggca |
| 901 | tactagtaca agtggtaatt tttgtacatt acactaaatt attagcattt gttttagcat |
| 961 | tacctaattt ttttcctgct ccatgcagac tgttagcttt taccttaaat gcttatttta |
| 1021 | aaatgacagt ggaagttttt ttttcctcta agtgccagta ttcccagagt tttggttttt |
| 1081 | gaactagcaa tgcctgtgaa aaagaaactg aatacctaag atttctgtct tggggttttt |
| 1141 | ggtgcatgca gttgattact tcttattttt cttaccaatt gtgaatgttg gtgtgaaaca |
| 1201 | aattaatgaa gcttttgaat catccctatt ctgtgtttta tctagtcaca taaatggatt |
| 1261 | aattactaat ttcagttgag accttctaat tggtttttac tgaaacattg agggaacaca |
| 1321 | aatttatggg cttcctgatg atgattcttc taggcatcat gtcctatagt ttgtcatccc |
| 1381 | tgatgaatgt aaagttacac tgttcacaaa ggttttgtct cctttccact gctattagtc |
| 1441 | atggtcactc tccccaaaat attatatttt ttctataaaa agaaaaaaat ggaaaaaaat |
| 1501 | tacaaggcaa tggaaactat tataaggcca tttccttttc acattagata aattactata |
| 1561 | aagactccta atagcttttc ctgttaaggc agacccagta tgaaatgggg attattatag |
| 1621 | caaccatttt ggggctatat ttacatgcta ctaaattttt ataataattg aaaagatttt |
| 1681 | aacaagtata aaaaattctc ataggaatta aatgtagtct ccctgtgtca gactgctctt |
| 1741 | tcatagtata actttaaatc ttttcttcaa cttgagtctt tgaagatagt tttaattctg |
| 1801 | cttgtgacat taaaagatta tttgggccag ttatagctta ttaggtgttg aagagaccaa |
| 1861 | ggttgcaagg ccaggccctg tgtgaacctt tgagctttca tagagagttt cacagcatgg |
| 1921 | actgtgtccc cacggtcatc cagtgttgtc atgcattggt tagtcaaaat ggggagggac |
| 1981 | tagggcagtt tggatagctc aacaagatac aatctcactc tgtggtggtc ctgctgacaa |
| 2041 | atcaagagca ttgcttttgt ttcttaagaa aacaaactct tttttaaaaa ttacttttaa |
| 2101 | atattaactc aaaagttgag attttggggt ggtggtgtgc caagacatta attttttttt |
| 2161 | taaacaatga agtgaaaaag ttttacaatc tctaggtttg gctagttctc ttaacactgg |
| 2221 | ttaaattaac attgcataaa cacttttcaa gtctgatcca tatttaataa tgctttaaaa |
| 2281 | taaaaataaa aacaatcctt ttgataaatt taaaatgtta cttattttaa aataaatgaa |
| 2341 | gtgagatggc atggtgaggt gaaagtatca ctggactagg aagaaggtga cttaggttct |
| 2401 | agataggtgt cttttaggac tctgattttg aggacatcac ttactatcca tttcttcatg |
| 2461 | ttaaaagaag tcatctcaaa ctcttagttt ttttttttta caactatgta atttatattc |
| 2521 | catttacata aggatacact tatttgtcaa gctcagcaca atctgtaaat ttttaaccta |
| 2581 | tgttacacca tcttcagtgc cagtcttggg caaaattgtg caagaggtga agtttatatt |
| 2641 | tgaatatcca ttctcgtttt aggactcttc ttccatatta gtgtcatctt gcctccctac |
| 2701 | cttccacatg ccccatgact tgatgcagtt ttaatacttg taattcccct aaccataaga |
| 2761 | tttactgctg ctgtggatat ctccatgaag ttttcccact gagtcacatc agaaatgccc |
| 2821 | tacatcttat ttcctcaggg ctcaagagaa tctgacagat accataaagg gatttgacct |
| 2881 | aatcactaat tttcaggtgg tggctgatgc tttgaacatc tctttgctgc ccaatccatt |
| 2941 | agcgacagta ggatttttca aacctggtat gaatagacag aaccctatcc agtggaagga |
| 3001 | gaatttaata aagatagtgc tgaaagaatt ccttaggtaa tctataacta ggactactcc |
| 3061 | tggtaacagt aatacattcc attgttttag taaccagaaa tcttcatgca atgaaaaata |
| 3121 | ctttaattca tgaagcttac tttttttttt tggtgtcaga gtctcgctct tgtcacccag |
| 3181 | gctggaatgc agtggcgcca tctcagctca ctgcaacctc catctcccag gttcaagcga |
| 3241 | ttctcgtgcc tcggcctcct gagtagctgg gattacaggc gtgtgccact acactcaact |
| 3301 | aatttttgta tttttaggag agacggggtt tcaccctgtt ggccaggctg gtctcgaact |
| 3361 | cctgacctca agtgatGcac ccaccttggc ctcataaacc tgttttgcag aactcattta |
| 3421 | ttcagcaaat atttattgag tgcctaccag atgccagtca ccgcacaagg cactgggtat |
| 3481 | atggtatccc caaacaagag acataatccc ggtccttagg tagtgctagt gtggtctgta |
| 3541 | atatcttact aaggcctttg gtatacgacc cagagataac acgatgcgta ttttagtttt |
| 3601 | gcaaagaagg ggtttggtct ctgtgccagc tctataattg ttttgctacg attccactga |
| 3661 | aactcttcga tcaagctact ttatgtaaat cacttcattg ttttaaagga ataaacttga |
| 3721 | ttatattgtt tttttatttg gcataactgt gattctttta ggacaattac tgtacacatt |
| 3781 | aaggtgtatg tcagatattc atattgaccc aaatgtgtaa tattccagtt ttctctgcat |
| 3841 | aagtaattaa aatatactta aaaattaata gttttatctg ggtacaaata aacaggtgcc |
| 3901 | tgaactagtt cacagacaag gaaacttcta tgtaaaaatc actatgattt ctgaattgct |
| 3961 | atgtgaaact acagatcttt ggaacactgt ttaggtaggg tgttaagact tacacagtac |
| 4021 | ctcgtttcta cacagagaaa gaaatggcca tacttcagga actgcagtgc ttatgagggg |
| 4081 | atatttaggc ctcttgaatt tttgatgtag atgggcattt ttttaaggta gtggttaatt |
| 4141 | acctttatgt gaactttgaa tggtttaaca aaagatttgt ttttgtagag attttaaagg |
| 4201 | gggagaattc tagaaataaa tgttacctaa ttattacagc cttaaagaca aaaatccttg |
| 4261 | ttgaagtttt tttaaaaaaa gctaaattac atagacttag gcattaacat gtttgtggaa |
| 4321 | gaatatagca gacgtatatt gtatcatttg agtgaatgtt cccaagtagg cattctaggc |
| 4381 | tctatttaac tgagtcacac tgcataggaa tttagaacct aacttttata ggttatcaaa |
| 4441 | actgttgtca ccattgcaca attttgtcct aatatataca tagaaacttt gtggggcatg |
| 4501 | ttaagttaca gtttgcacaa gttcatctca tttgtattcc attgattttt tttttcttct |
| 4561 | aaacattttt tcttcaaaca gtatataact ttttttaggg gatttttttt tagacagcaa |
| 4621 | aaactatctg aagatttcca tttgtcaaaa agtaatgatt tcttgataat tgtgtagtaa |
| 4681 | tgttttttag aacccagcag ttaccttaaa gctgaattta tatttagtaa cttctgtgtt |
| 4741 | aatactggat agcatgaatt ctgcattgag aaactgaata gctgtcataa aatgaaactt |
| 4801 | tctttctaaa gaaagatact cacatgagtt cttgaagaat agtcataact agattaagat |
| 4861 | ctgtgtttta gtttaatagt ttgaagtgcc tgtttgggat aatgataggt aatttagatg |
| 4921 | aatttagggg aaaaaaaagt tatctgcaga tatgttgagg gcccatctct ccccccacac |
| 4981 | ccccacagag ctaactgggt tacagtgttt tatccgaaag tttccaattc cactgtcttg |
| 5041 | tgttttcatg ttgaaaatac ttttgcattt ttcctttgag tgccaatttc ttactagtac |
| 5101 | tatttcttaa tgtaacatgt ttacctggaa tgtattttaa ctatttttgt atagtgtaaa |
| 5161 | ctgaaacatg cacattttgt acattgtgct ttcttttgtg ggacatatgc agtgtgatcc |
| 5221 | agttgttttc catcatttgg ttgcgctgac ctaggaatgt tggtcatatc aaacattaaa |
| 5281 | aatgaccact cttttaattg aaattaactt ttaaatgttt ataggagtat gtgctgtgaa |
| 5341 | gtgatctaaa atttgtaata tttttgtcat gaactgtact actcctaatt attgtaatgt |
| 5401 | aataaaaata gttacagtga caaaaaaaaa aaaaaa |

Human KRAS, transcript variant b, comprising the LCS6 SNP, is encoded by the following mRNA sequence (SEQ ID NO: 4) (untranslated regions are bolded, LCS6 is underlined. SNP is capitalized):

| | |
|---|---|
| 1 | ggccgcggcg gcggaggcag cagcggcggc ggcagtggcg gcggcgaagg tggcggcggc |
| 61 | tcggccagta ctcccggccc ccgccatttc ggactgggag cgagcgcggc gcaggcactg |
| 121 | aaggcggcgg cggggccaga ggctcagcgg ctcccaggtg cgggagagag gcctgctgaa |
| 181 | aatgactgaa tataaacttg tggtagttgg agctggtggc gtaggcaaga gtgccttgac |
| 241 | gatacagcta attcagaatc attttgtgga cgaatatgat ccaacaatag aggattccta |
| 301 | caggaagcaa gtagtaattg atggagaaac ctgtctcttg gatattctcg acacagcagg |
| 361 | tcaagaggag tacagtgcaa tgagggacca gtacatgagg actggggagg gctttctttg |
| 421 | tgtatttgcc ataaataata ctaaatcatt tgaagatatt caccattata gagaacaaat |
| 481 | taaaagagtt aaggactctg aagatgtacc tatggtccta gtaggaaata aatgtgattt |
| 541 | gccttctaga acagtagaca caaaacaggc tcaggactta gcaagaagtt atggaattcc |
| 601 | ttttattgaa acatcagcaa agacaagaca gggtgttgat gatgccttct atacattagt |
| 661 | tcgagaaatt cgaaaacata aagaaaagat gagcaaagat ggtaaaaaga agaaaaagaa |
| 721 | gtcaaagaca aagtgtgtaa ttatgtaaat acaatttgta cttttttctt aaggcatact |
| 781 | agtacaagtg gtaatttttg tacattacac taaattatta gcatttgttt tagcattacc |
| 841 | taattttttt cctgctccat gcagactgtt agcttttacc ttaaatgctt attttaaaat |
| 901 | gacagtggaa gttttttttt cctctaagtg ccagtattcc cagagttttg gtttttgaac |
| 961 | tagcaatgcc tgtgaaaaag aaactgaata cctaagattt ctgtcttggg gtttttggtg |
| 1021 | catgcagttg attacttctt atttttctta ccaattgtga atgttggtgt gaaacaaatt |
| 1081 | aatgaagctt ttgaatcatc cctattctgt gttttatcta gtcacataaa tggattaatt |
| 1141 | actaatttca gttgagacct tctaattggt ttttactgaa acattgaggg aacacaaatt |
| 1201 | tatgggcttc ctgatgatga ttcttctagg catcatgtcc tatagtttgt catccctgat |
| 1261 | gaatgtaaag ttacactgtt cacaaaggtt ttgtctcctt tccactgcta ttagtcatgg |
| 1321 | tcactctccc caaaatatta tattttttct ataaaaagaa aaaaatggaa aaaaattaca |
| 1381 | aggcaatgga aactattata aggccatttc cttttcacat tagataaatt actataaaga |
| 1441 | ctcctaatag cttttcctgt taaggcagac ccagtatgaa atggggatta ttatagcaac |
| 1501 | cattttgggg ctatatttac atgctactaa atttttataa taattgaaaa gattttaaca |
| 1561 | agtataaaaa attctcatag gaattaaatg tagtctccct gtgtcagact gctctttcat |
| 1621 | agtataactt taaatctttt cttcaacttg agtctttgaa gatagtttta attctgcttg |
| 1681 | tgacattaaa agattatttg ggccagttat agcttattag gtgttgaaga gaccaaggtt |
| 1741 | gcaaggccag gccctgtgtg aacctttgag ctttcataga gagtttcaca gcatggactg |
| 1801 | tgtccccacg gtcatccagt gttgtcatgc attggttagt caaaatgggg agggactagg |
| 1861 | gcagtttgga tagctcaaca agatacaatc tcactctgtg gtggtcctgc tgacaaatca |
| 1921 | agagcattgc ttttgtttct taagaaaaca aactcttttt taaaaattac ttttaaatat |
| 1981 | taactcaaaa gttgagattt tggggtggtg gtgtgccaag acattaattt tttttttaaa |
| 2041 | caatgaagtg aaaaagtttt acaatctcta ggtttggcta gttctcttaa cactggttaa |
| 2101 | attaacattg cataaacact tttcaagtct gatccatatt taataatgct ttaaaataaa |
| 2161 | aataaaaaca atccttttga taaatttaaa atgttactta ttttaaaata aatgaagtga |
| 2221 | gatggcatgg tgaggtgaaa gtatcactgg actaggaaga aggtgactta ggttctagat |
| 2281 | aggtgtcttt taggactctg attttgagga catcacttac tatccatttc ttcatgttaa |
| 2341 | aagaagtcat ctcaaactct tagttttttt tttttacaac tatgtaattt atattccatt |
| 2401 | tacataagga tacacttatt tgtcaagctc agcacaatct gtaaattttt aacctatgtt |
| 2461 | acaccatctt cagtgccagt cttgggcaaa attgtgcaag aggtgaagtt tatatttgaa |
| 2521 | tatccattct cgttttagga ctcttcttcc atattagtgt catcttgcct ccctaccttc |
| 2581 | cacatgcccc atgacttgat gcagttttaa tacttgtaat tcccctaacc ataagattta |
| 2641 | ctgctgctgt ggatatctcc atgaagtttt cccactgagt cacatcagaa atgccctaca |
| 2701 | tcttatttcc tcagggctca agagaatctg acagatacca taaagggatt tgacctaatc |
| 2761 | actaattttc aggtggtggc tgatgctttg aacatctctt tgctgcccaa tccattagcg |
| 2821 | acagtaggat ttttcaaacc tggtatgaat agacagaacc ctatccagtg gaaggagaat |
| 2881 | ttaataaaga tagtgctgaa agaattcctt aggtaatcta taactaggac tactcctggt |
| 2941 | aacagtaata cattccattg ttttagtaac cagaaatctt catgcaatga aaaatacttt |
| 3001 | aattcatgaa gcttactttt tttttttggt gtcagagtct cgctcttgtc acccaggctg |
| 3061 | gaatgcagtg gcgccatctc agctcactgc aacctccatc tcccaggttc aagcgattct |
| 3121 | cgtgcctcgg cctcctgagt agctgggatt acaggcgtgt gccactacac tcaactaatt |
| 3181 | tttgtatttt taggagagac ggggtttcac cctgttggcc aggctggtct cgaactcctg |
| 3241 | acctcaagtg atGcacccac cttggcctca taaacctgtt ttgcagaact catttattca |
| 3301 | gcaaatattt attgagtgcc taccagatgc cagtcaccgc acaaggcact gggtatatgg |
| 3361 | tatccccaaa caagagacat aatcccggtc cttaggtagt gctagtgtgg tctgtaatat |
| 3421 | cttactaagg cctttggtat acgacccaga gataacacga tgcgtatttt agttttgcaa |
| 3481 | agaaggggtt tggtctctgt gccagctcta taattgtttt gctacgattc cactgaaact |
| 3541 | cttcgatcaa gctactttat gtaaatcact tcattgtttt aaaggaataa acttgattat |
| 3601 | attgtttttt tatttggcat aactgtgatt cttttaggac aattactgta cacattaagg |
| 3661 | tgtatgtcag atattcatat tgacccaaat gtgtaatatt ccagttttct ctgcataagt |
| 3721 | aattaaaata tacttaaaaa ttaatagttt tatctgggta caaataaaca ggtgcctgaa |
| 3781 | ctagttcaca gacaaggaaa cttctatgta aaaatcacta tgatttctga attgctatgt |
| 3841 | gaaactacag atctttggaa cactgtttag gtagggtgtt aagacttaca cagtacctcg |
| 3901 | tttctacaca gagaaagaaa tggccatact tcaggaactg cagtgcttat gaggggatat |
| 3961 | ttaggcctct tgaatttttg atgtagatgg gcattttttt aaggtagtgg ttaattacct |
| 4021 | ttatgtgaac tttgaatggt ttaacaaaag atttgttttt gtagagattt taaaggggga |
| 4081 | gaattctaga aataaatgtt acctaattat tacagcctta aagacaaaaa tccttgttga |
| 4141 | agttttttta aaaaaagcta aattacatag acttaggcat taacatgttt gtggaagaat |
| 4201 | atagcagacg tatattgtat catttgagtg aatgttccca agtaggcatt ctaggctcta |
| 4261 | tttaactgag tcacactgca taggaattta gaacctaact tttataggtt atcaaaactg |
| 4321 | ttgtcaccat tgcacaattt tgtcctaata tatacataga aactttgtgg ggcatgttaa |
| 4381 | gttacagttt gcacaagttc atctcatttg tattccattg attttttttt tcttctaaac |
| 4441 | attttttctt caaacagtat ataacttttt ttaggggatt tttttttaga cagcaaaaac |
| 4501 | tatctgaaga tttccatttg tcaaaaagta atgatttctt gataattgtg tagtaatgtt |
| 4561 | ttttagaacc cagcagttac cttaaagctg aatttatatt tagtaacttc tgtgttaata |
| 4621 | ctggatagca tgaattctgc attgagaaac tgaatagctg tcataaaatg aaactttctt |
| 4681 | tctaaagaaa gatactcaca tgagttcttg aagaatagtc ataactagat taagatctgt |
| 4741 | gttttagttt aatagtttga agtgcctgtt tgggataatg ataggtaatt tagatgaatt |
| 4801 | taggggaaaa aaaagttatc tgcagatatg ttgagggccc atctctcccc ccacaccccc |
| 4861 | acagagctaa ctgggttaca gtgttttatc cgaaagtttc caattccact gtcttgtgtt |
| 4921 | ttcatgttga aaatactttt gcatttttcc tttgagtgcc aatttcttac tagtactatt |
| 4981 | tcttaatgta acatgtttac ctggaatgta ttttaactat ttttgtatag tgtaaactga |
| 5041 | aacatgcaca ttttgtacat tgtgctttct tttgtgggac atatgcagtg tgatccagtt |
| 5101 | gttttccatc atttggttgc gctgacctag gaatgttggt catatcaaac attaaaaatg |
| 5161 | accactcttt taattgaaat taacttttaa atgtttatag gagtatgtgc tgtgaagtga |
| 5221 | tctaaaattt gtaatatttt tgtcatgaac tgtactactc ctaattattg taatgtaata |
| 5281 | aaaatagtta cagtgacaaa aaaaaaaaaa aa |

### Modified Oligonucleotides

As used herein, the term “let-7 complementary site” is meant to describe any region of a gene or gene transcript that binds a member of the let-7 family of miRNAs. Moreover, this term encompasses those sequences within a gene or gene transcript that are complementary to the sequence of a let-7 family miRNA.

The Human KRAS 3′ UTR comprises 8 LCSs named LCS1-LCS8, respectively. For the following sequences, thymine (T) may be substituted for uracil (U). LCS1 comprises the sequence GACAGUGGAAGUUUUUUUUUCCUCG (SEQ ID NO: 5). LCS2 comprises the sequence AUUAGUGUCAUCUUGCCUC (SEQ ID NO: 6). LCS3 comprises the sequence AAUGCCCUACAUCUUAUUUUCCUCA (SEQ ID NO: 7). LCS4 comprises the sequence GGUUCAAGCGAUUCUCGUGCCUCG (SEQ ID NO: 8). LCS5 comprises the sequence GGCUGGUCCGAACUCCUGACCUCA (SEQ ID NO: 9). LCS6 comprises the sequence GAUUCACCCACCUUGGCCUCA (SEQ ID NO: 10). LCS7 comprises the sequence GGGUGUUAAGACUUGACACAGUACCUCG (SEQ ID NO: 11). LCS8 comprises the sequence AGUGCUUAUGAGGGGAUAUUUAGGCCUC (SEQ ID NO: 12).

The present invention provides compositions containing modified oligos that bind to the LCS6 SNP defined by the sequence GAUGCACCCACCUUGGCCUCA (SNP bolded for emphasis) (SEQ ID NO: 13). In a preferred embodiment the modified oligo is derived from let-7b. For the preceding sequence, thymine (T) may be substituted for uracil (U).

The invention provides compositions comprising modified oligos that are derived from wild type let-7 family miRNAs. Exemplary let-7 miRNAs include, but are not limited to, let-7a, let-7b, let-7c, let-7d, let-7e, let-7f let-7g, let-7h, and let-7i. For the following sequences, thymine (T) may be substituted for uracil (U). let-7a comprises the sequence UUGAUAUGUUGGAUGAUGGAGU (SEQ ID NO: 14). let-7b comprises the sequence UUGGUGUGUUGGAUGAUGGAGU (SEQ ID NO: 15). let-7c comprises the sequence UUGGUAUGUUGGAUGAUGGAGU (SEQ ID NO: 16). let-7d comprises the sequence UGAUACGUUGGAUGAUGGAGA (SEQ ID NO: 17). let-7e comprises the sequence UAUAUGUUGGAGGAUGGAGU (SEQ ID NO: 18). let-7f comprises the sequence UUGAUAUGUUAGAUGAUGGAGU (SEQ ID NO: 19). let-7g comprises the sequence GACAUGUUUGAUGAUGGAGU (SEQ ID NO: 20). let-7i comprises the sequence UGUCGUGUUUGUUGAUGGAGU (SEQ ID NO: 21).

The present invention provides isolated nucleic acids and compositions containing modified oligo molecules including, but not limited to, the preferred modified oligo defined by the sequence UGAGGUAGUAGGUUGUGUGCUUUU (variant nucleic acid residue at position 20 bolded) (SEQ ID NO: 22). For the preceding sequence, thymine (T) may be substituted for uracil (U).

### Isolated Nucleic Acid Molecules

The present invention provides isolated nucleic acid molecules that bind sequences containing presumptive miRNA binding sites, e.g. modified oligonucleotides (referred to herein as “oligos”). These miRNA binding sites contain one or more mutations. In certain aspects of the invention, these mutations are SNPs. Exemplary isolated nucleic acid molecules containing one or more SNPs include, but are not limited to, the nucleic acid molecules of SEQ ID NOs: 3, 4, and 13. Isolated nucleic acid molecules containing one or more SNPs disclosed herein may be interchangeably referred to throughout the present text as “SNP-containing nucleic acid molecules”. In one aspect of the invention, isolated nucleic acid molecules, modified oligos, are engineered to bind SNP-containing nucleic acid molecules. In other aspects of the invention, isolated nucleic acid molecules, such as modified oligos, bind sequences containing one or more insertions, deletions, inversions, frameshifts, translocations, recombinations, or substitutions.

The isolated nucleic acid molecules of the present invention include single- and double-stranded ribonucleic acid (RNA) and deoxyribonucleic acid (DNA) molecules, as well as all art-recognized analogs, derivatives, or hybrid molecules thereof. Isolated nucleic acid molecules of the present invention also include reagents for synthesizing modified oligos, such as isolated full-length genes, transcripts, cDNA molecules, primers, vectors, plasmids, endogenous or naturally-occurring miRNAs, and fragments thereof.

As used herein, an “isolated nucleic acid molecule” generally is one that binds a miRNA binding site or miRNA complementary site. Isolated nucleic acid molecules of the present invention are engineered to bind sequences containing one or more mutations that alter or modify the binding efficacy of at least one endogenous and/or naturally occurring miRNA, and is separated from most other nucleic acids present in the natural source of the nucleic acid molecule. Moreover, an “isolated” nucleic acid molecule can be substantially free of other cellular material, or culture medium when produced by recombinant techniques, or chemical precursors or other chemicals when chemically synthesized. A nucleic acid molecule can be fused to other coding or regulatory sequences and still be considered “isolated”. Nucleic acid molecules present in non-human transgenic animals, which do not naturally occur in the animal, are also considered “isolated”. For example, recombinant nucleic acid molecules contained in a vector are considered “isolated”. Further examples of “isolated” nucleic acid molecules include recombinant DNA or RNA molecules maintained in heterologous host cells, and purified (partially or substantially) DNA or RNA molecules in solution. Isolated RNA molecules include in vivo or in vitro RNA transcripts of the isolated nucleic acid molecules of the present invention. Moreover, isolated RNA molecules include, but are not limited to, messenger RNA (mRNA), interfering RNA (RNAi), short interfering RNA (siRNA), short hairpain RNA (shRNA), double-stranded RNA (dsRNA), and microRNA (miRNA). Isolated nucleic acid molecules according to the present invention further include such molecules produced synthetically.

Generally, an isolated nucleic acid molecule comprises one or more sequences engineered to bind to a miRNA binding site containing one or more mutations, with flanking nucleotide sequences on either side of the modified oligo sequence. A flanking sequence can include nucleotide residues that are naturally associated with the targeted miRNA binding site and/or heterologous nucleotide sequences. Preferably the flanking sequence is up to about 500, 300, 100, 60, 50, 30, 25, 20, 15, 10, 8, or 4 nucleotides (or any other length in-between) on either side of the modified oligo sequence, or as long as the full-length gene, entire coding, or non-coding sequence (or any portion thereof, such as, an exon, intron, or a 5′ or 3′ untranslated region).

Isolated nucleic acids molecules of the invention are associated with, bound to, conjugated to, linked to, or incorporated with a virus (or any part or fragment thereof), a liposome, a lipid, an antibody, an intrabody, a protein, a receptor, a ligand, a cytotoxic compound, a radioisotope, a toxin, a chemotherapeutic agent, a salt, an ester, a prodrug, a polymer, a hydrogel, a microcapsule, a nanocapsule, a microsphere, a cyclodextin, a plasmid, an expression vector, a proteinaceous vector, a detectable label (e.g. fluorescent, radioactive, magnetic, paramagnetic, etc.), an antigen, a diluent, an excipient, an adjuvant, an emulsifier, a buffer, a stabilizer, or a preservative.

As used herein, the term “fragment” is meant to describe an isolated nucleic acid molecule that is shorter the isolated nucleic acid molecule from which it is derived. Fragments of isolated nucleic acid molecules of the invention can contain, consist of, or comprise any part of the isolated nucleic acid molecule from which it is derived. A fragment typically comprises a contiguous nucleotide sequence at least about 8 or more nucleotides, more preferably at least about 10 or more nucleotides, and even more preferably at least about 16 or more nucleotides. Further, a fragment could comprise at least about 18, 20, 21, 22, 25, 30, 40, 50, 60, 100, 250 or 500 (or any other number in-between) nucleotides in length. The length of the fragment will be based on its intended use. A labeled probe can then be used, for example, to screen a cDNA library, genomic DNA library, or mRNA to isolate nucleic acid corresponding to the region of interest. Further, primers can be used in amplification reactions, such as for purposes of assaying one or more miRNA binding sites or for cloning specific regions of a gene.

An isolated nucleic acid molecule of the present invention further encompasses a modified or synthetic oligo that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195; and 4,683,202; PCR Technology: Principles and Applications for DNA Amplification, ed. H. A. Erlich, Freeman Press, NY, N.Y., 1992), ligase chain reaction (LCR) (Wu and Wallace, Genomics 4:560, 1989; Landegren et al., Science 241:1077, 1988), strand displacement amplification (SDA) (U.S. Pat. Nos. 5,270,184; and 5,422,252), transcription-mediated amplification (TMA) (U.S. Pat. No. 5,399,491), linked linear amplification (LLA) (U.S. Pat. No. 6,027,923), and the like, and isothermal amplification methods such as nucleic acid sequence based amplification (NASBA), and self-sustained sequence replication (Guatelli et al., Proc. Natl. Acad. Sci. USA 87: 1874, 1990). Based on such methodologies, a person skilled in the art can readily design primers in any suitable regions 5′ and 3′ to a SNP disclosed herein.

As used herein, an “amplified polynucleotide” of the invention is a isolated nucleic acid molecule whose amount has been increased at least two fold by any nucleic acid amplification method performed in vitro as compared to its starting amount in a test sample. In other preferred embodiments, an amplified polynucleotide is the result of at least ten fold, fifty fold, one hundred fold, one thousand fold, or even ten thousand fold increase as compared to its starting amount in a test sample. In a typical PCR amplification, a polynucleotide of interest is often amplified at least fifty thousand fold in amount over the unamplified genomic DNA, but the precise amount of amplification needed for an assay depends on the sensitivity of the subsequent detection method used.

Generally, an amplified polynucleotide is at least about 10 nucleotides in length. More typically, an amplified polynucleotide is at least about 16 nucleotides in length. In a preferred embodiment of the invention, an amplified polynucleotide is at least about 2025 nucleotides in length. In a more preferred embodiment of the invention, an amplified polynucleotide is at least about 21, 22, 23, 24, 25, 30, 35, 40, 45, 50, or 60 nucleotides in length. In yet another preferred embodiment of the invention, an amplified polynucleotide is at least about 100, 200, or 300 nucleotides in length. While the total length of an amplified polynucleotide of the invention can be as long as an exon, an intron, a 5′ UTR, a 3′ UTR, or the entire gene where the altered miRNA binding site of interest resides, an amplified product is typically no greater than about 1,000 nucleotides in length (although certain amplification methods may generate amplified products greater than 1000 nucleotides in length). More preferably, an amplified polynucleotide is not greater than about 600 nucleotides in length.

In a specific embodiment of the invention, the amplified product is at least about 24 nucleotides in length, and binds a SNP-containing let-7 complementary site (LCS). In a specific embodiment, the amplified product is at least about 24 nucleotides in length, and comprises SEQ ID NO: 22. Such a product may have additional sequences on its 5′ end or 3′ end or both.

The present invention provides isolated nucleic acid molecules that comprise, consist of, or consist essentially of one or more polynucleotide sequences that bind mutated or altered miRNA binding sites and/or fragments thereof.

Accordingly, the present invention provides nucleic acid molecules that consist of the nucleotide sequence of SEQ ID NOs: 22-36. A nucleic acid molecule consists of a nucleotide sequence when the nucleotide sequence is the complete nucleotide sequence of the nucleic acid molecule.

The present invention further provides nucleic acid molecules that consist essentially of the nucleotide sequence of SEQ ID NOs: 22-36. A nucleic acid molecule consists essentially of a nucleotide sequence when such a nucleotide sequence is present with only a few additional nucleotide residues in the final nucleic acid molecule.

The present invention further provides nucleic acid molecules that comprise the nucleotide sequence of SEQ ID NOs: 22-36. A nucleic acid molecule comprises a nucleotide sequence when the nucleotide sequence is at least part of the final nucleotide sequence of the nucleic acid molecule. In such a fashion, the nucleic acid molecule can be only the nucleotide sequence or have additional nucleotide residues, such as residues that are naturally associated with it or heterologous nucleotide sequences. Such a nucleic acid molecule can have one to a few additional nucleotides or can comprise many more additional nucleotides. A brief description of how various types of these nucleic acid molecules can be readily made and isolated is provided below, and such techniques are well known to those of ordinary skill in the art (Sambrook and Russell, 2000, Molecular Cloning: A Laboratory Manual, Cold Spring Harbor Press, NY).

The isolated nucleic acid molecules include, but are not limited to, nucleic acid molecules having a sequence encoding a peptide alone, a sequence encoding a mature peptide and additional coding sequences such as a leader or secretory sequence (e.g., a pre-pro or pro-protein sequence), a sequence encoding a mature peptide with or without additional coding sequences, plus additional non-coding sequences, for example introns and non-coding 5′ and 3′ sequences such as transcribed but untranslated sequences that play a role in, for example, transcription, mRNA processing (including splicing and polyadenylation signals), ribosome binding, and/or stability of mRNA. In addition, the nucleic acid molecules may be fused to heterologous marker sequences encoding, for example, a peptide that facilitates purification.

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof (Sambrook and Russell, 2000, Molecular Cloning: A Laboratory Manual, Cold Spring Harbor Press, NY). Furthermore, isolated nucleic acid molecules can also be partially or completely in the form of one or more types of nucleic acid analogs, such as peptide nucleic acid (PNA) (U.S. Pat. Nos. 5,539,082; 5,527,675; 5,623,049; 5,714,331). The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art (see, e.g., Corey, “Peptide nucleic acids: expanding the scope of nucleic acid recognition”, Trends Biotechnol. 1997 June; 15(6):224-9, and Hyrup et al., “Peptide nucleic acids (PNA): synthesis, properties and potential applications”, Bioorg Med. Chem. 1996 January; 4(1):5-23). Furthermore, large-scale automated oligonucleotide/PNA synthesis (including synthesis on an array or bead surface or other solid support) can readily be accomplished using commercially available nucleic acid synthesizers, such as the Applied Biosystems (Foster City, Calif.) 3900 High-Throughput DNA Synthesizer or Expedite 8909 Nucleic Acid Synthesis System, and the sequence information provided herein.

The present invention encompasses nucleic acid analogs that contain modified, synthetic, or non-naturally occurring nucleotides or structural elements or other alternative/modified nucleic acid chemistries known in the art. Such nucleic acid analogs are useful, for example, as detection reagents (e.g., primers/probes). Furthermore, kits/systems (such as beads, arrays, etc.) that include these analogs are also encompassed by the present invention. For example, PNA oligomers that are based on the polymorphic sequences of the present invention are specifically contemplated. PNA oligomers are analogs of DNA in which the phosphate backbone is replaced with a peptide-like backbone (Lagriffoul et al., Bioorganic & Medicinal Chemistry Letters, 4: 1081-1082 (1994), Petersen et al., Bioorganic & Medicinal Chemistry Letters, 6: 793-796 (1996), Kumar et al., Organic Letters 3(9): 1269-1272 (2001), WO96/04000). PNA hybridizes to complementary RNA or DNA with higher affinity and specificity than conventional oligonucleotides and oligonucleotide analogs. The properties of PNA enable novel molecular biology and biochemistry applications unachievable with traditional oligonucleotides and peptides.

The term “modified or synthetic oligonucleotide (oligo) molecule” is not limited to molecules containing only naturally-occurring RNA or DNA, but also encompasses chemically-modified nucleotides and non-nucleotides.

In certain embodiments, the modified oligo molecules lack 2′-hydroxy (2′-OH) containing nucleotides. In certain embodiments modified oligos do not require the presence of nucleotides having a 2′-hydroxy group for mediating gene silencing and as such, isolated nucleic acid molecules, e.g. modified oligos, optionally do not include any ribonucleotides (e.g., nucleotides having a 2′-OH group). Such oligo molecules that do not require the presence of ribonucleotides within the oligo molecule to support gene silencing can however have an attached linker or linkers or other attached or associated groups, moieties, or chains containing one or more nucleotides with 2′0H groups. Optionally, miRNA molecules can comprise ribonucleotides at about 5, 10, 20, 30, 40, or 50% of the nucleotide positions.

As used herein, the term “modified oligo” is meant to be equivalent to other terms used to describe nucleic acid molecules that are capable of mediating sequence specific gene silencing or interference, e.g., microRNA (miRNA), short interfering RNA (siRNA), double-stranded RNA (dsRNA), interfering RNA (RNAi), short hairpin RNA (shRNA), short interfering oligonucleotide, short interfering nucleic acid, short interfering modified oligonucleotide, chemically-modified siRNA, post-transcriptional gene silencing RNA (ptgsRNA), and other art-recognized equivalents. As used herein, the term “gene silencing” is meant to describe the downregulation, knock-down, degradation, inhibition, suppression, repression, prevention, or decreased expression of a gene, transcript and/or polypeptide product. Gene silencing and interference also describe the prevention of translation of mRNA transcripts into a polypeptide. Translation is prevented, inhibited, or decreased by degrading mRNA transcripts or blocking mRNA translation.

In other embodiments, modified oligo molecules, or precursors thereof, may comprise separate sense and antisense sequences or regions, wherein the sense and antisense regions are covalently linked by nucleotide or non-nucleotide linker molecules, or are alternately non-covalently linked by ionic interactions, hydrogen bonding, van der waals interactions, hydrophobic interactions, and/or stacking interactions.

As used herein the term “antisense RNA” is an RNA strand having a sequence complementary to a target gene mRNA, and thought to induce gene silencing or interference by binding to the target gene mRNA. As used herein the term “Sense RNA” has a sequence complementary to the antisense RNA, and when annealed to its complementary antisense RNA, forms a siRNA. Antisense and sense RNAs are conventionally synthesized with an RNA synthesizer.

Modified oligos are assembled from two separate oligonucleotides, where one strand is the sense strand and the other is the antisense strand, wherein the antisense and sense strands are self-complementary (i.e., each strand comprises nucleotide sequence that is complementary to nucleotide sequence in the other strand; such as where the antisense strand and sense strand form a duplex or double stranded structure, e.g., wherein the double stranded region is about 1, 2, 5, 10, 15, or 19 base pairs, or any value in between). The antisense strand may comprise a nucleotide sequence that is complementary to a nucleotide sequence in a target nucleic acid molecule or a portion thereof, and the sense strand may comprise a nucleotide sequence corresponding to the target nucleic acid sequence or a portion thereof. Alternatively, the modified oligo is assembled from a single oligonucleotide, where the self-complementary sense and antisense regions of the oligo are linked by means of a nucleic acid-based or non-nucleic acid-based linker(s). The modified oligo is assembled as a single oligonucleotide representing the antisense strand.

In certain embodiments, modified oligos engineered for intracellular delivery according to the instant methods and compositions include a polynucleotide with a duplex, asymmetric duplex, hairpin or asymmetric hairpin secondary structure, having self-complementary sense and antisense regions, wherein the antisense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

Non-limiting examples of chemical modifications that are made in a modified oligo include without limitation phosphorothioate internucleotide linkages, 2′-deoxyribonucleotides, 2′-0-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation. These chemical modifications, when used in modified oligos, preserve silencing or interference activity in cells while at the same time, dramatically increasing the serum stability of these compounds.

In a non-limiting example, the introduction of chemically-modified nucleotides into nucleic acid molecules provides a powerful tool in overcoming potential limitations of in vivo stability and bioavailability inherent to native RNA molecules that are delivered exogenously. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, e.g., when compared to an all-RNA nucleic acid molecule, the overall activity of the modified nucleic acid molecule can be greater than that of the native molecule due to improved stability and/or delivery of the molecule. Unlike native unmodified oligos, chemically-modified oligos can also minimize the possibility of activating interferon activity in humans.

The antisense region of a modified oligo molecule of the invention can comprise a phosphorothioate internucleotide linkage at the 3′-end of said antisense region. In any of the embodiments of modified oligo molecules described herein, the antisense region can comprise about one to about five phosphorothioate internucleotide linkages at the 5′-end of said antisense region. In any of the embodiments of modified oligo molecules described herein, the 3′-terminal nucleotide overhangs of a modified oligo molecule of the disclosure can comprise ribonucleotides or deoxyribonucleotides that are chemically-modified at a nucleic acid sugar, base, or backbone. In any of the embodiments of modified oligo molecules described herein, the 3′-terminal nucleotide overhangs can comprise one or more universal base ribonucleotides. In any of the embodiments of modified oligo molecules described herein, the 3′-terminal nucleotide overhangs can comprise one or more acyclic nucleotides.

For example, in a non-limiting example, a chemically-modified modified oligo may have about 1, 2, 3, 4, 5, 6, 7, 8 or more phosphorothioate internucleotide linkages in one variant miRNA strand. In yet another embodiment, a chemically-modified modified oligo individually may have about 1, 2, 3, 4, 5, 6, 7, 8 or more phosphorothioate internucleotide linkages in both variant miRNA strands. The phosphorothioate internucleotide linkages can be present in one or both oligonucleotide strands of the modified oligo duplex, e.g., in the sense strand, the antisense strand, or both strands. The modified oligo molecules of the invention can comprise one or more phosphorothioate internucleotide linkages at the 3′-end, the 5′-end, or both of the 3′- and 5′-ends of the sense strand, the antisense strand, or both strands. An exemplary modified oligo molecule can comprise about 1 to about 5 or more (e.g., about 1, 2, 3, 4, 5, or more) consecutive phosphorothioate internucleotide linkages at the 5′-end of the sense strand, the antisense strand, or both strands. In another non-limiting example, modified oligo molecules of the invention can comprise one or more (e.g., about 1, 2, 3, 4, 5, 6, 7, 8, 9, 10, or more) pyrimidine phosphorothioate internucleotide linkages in the sense strand, the antisense strand, or both strands. In yet another non-limiting example, modified oligo molecules of the invention can comprise one or more (e.g., about 1, 2, 3, 4, 5, 6, 7, 8, 9, 10, or more) purine phosphorothioate internucleotide linkages in the sense strand, the antisense strand, or both strands.

A modified oligo molecule may be comprised of a circular nucleic acid molecule, wherein the modified oligo is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

A circular modified oligo molecule contains two loop motifs, wherein one or both loop portions of the modified oligo molecule is biodegradable. For example, a circular modified oligo molecule of this disclosure is designed such that degradation of the loop portions of the modified oligo molecule in vivo can generate a double-stranded modified oligo molecule with 3′-terminal overhangs, such as 3′-terminal nucleotide overhangs comprising about 2 nucleotides.

Modified nucleotides present in modified oligo molecules, preferably in the antisense strand of the modified oligos, but also optionally in the sense and/or both antisense and sense strands, comprise modified nucleotides having properties or characteristics similar to naturally occurring ribonucleotides. For example, the invention provides modified oligo molecules including modified nucleotides having a northern conformation (e.g., northern pseudorotation cycle, see, e.g., Saenger, Principles of Nucleic Acid Structure, Springer-Verlag Ed., 1984). As such, chemically modified nucleotides present in the modified oligos of the invention, preferably in the antisense strand of the modified oligo molecules of the invention, but also optionally in the sense and/or both antisense and sense strands, are resistant to nuclease degradation while at the same time maintaining the capacity to mediate silencing or interference. Non-limiting examples of nucleotides having a northern configuration include locked nucleic acid (LNA) nucleotides (e.g., 2′-O, 4′-C-methylene-(D-ribofuranosyl) nucleotides); 2′-methoxyethoxy (MOE) nucleotides; 2′-methyl-thio-ethyl, 2′-deoxy-2′-fluoro nucleotides. 2′-deoxy-2′-chloro nucleotides, 2′-azido nucleotides, and 2′-0-methyl nucleotides.

The sense strand of a double stranded modified oligo molecule may have a terminal cap moiety such as an inverted deoxybasic moiety, at the 3′-end, 5′-end, or both 3′ and 5′-ends of the sense strand.

A modified oligo is further comprised of a nucleotide, non-nucleotide, or mixed nucleotide/non-nucleotide linker that joins the sense region of the modified oligo to the antisense region of the modified oligo. In one embodiment, a nucleotide linker can be a linker of >2 nucleotides in length, e.g., about 3, 4, 5, 6, 7, 8, 9, or 10 nucleotides in length. In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest. For example, the aptamer can be used to bind to a ligand binding domain of a protein, thereby preventing interaction of the naturally occurring ligand with the protein. This is a non-limiting example and those in the art will recognize that other embodiments can be readily generated using techniques generally known in the art. (See, e.g., Gold, et al, Annu. Rev. Biochem. 64:763, 1995; Brody and Gold, J. Biotechnol 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chemistry 45:1628, 1999.

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having from 2 to 100 ethylene glycol units). Specific examples include those described by Seela and Kaiser, Nucleic Acids Res. 18:6353, 1990 and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma, et al., Nucleic Acids Res. 21:2585, 1993 and Biochemistry 32:1751, 1993; Durand, et al., Nucleic Acids Res. 18:6353, 1990; McCurdy, et al., Nucleosides & Nucleotides 10:287, 1991; Jschke, et al., Tetrahedron Lett. 34:301, 1993; Ono, et al., Biochemistry 30:9914 (1991); Arnold, et al., International Publication No. WO 89/02439; Usman, et al., International Publication No. WO 95/06731; Dudycz, et al., International Publication No. WO 95/11910 and Ferentz and Verdine, J. Am. Chem. Soc. 113:4000, 1991. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymidine, e.g., at the C1 position of the sugar.

Additional examples of nucleic acid modifications that improve the binding properties and/or stability of a nucleic acid include the use of base analogs such as inosine, intercalators (U.S. Pat. No. 4,835,263) and the minor groove binders (U.S. Pat. No. 5,801,115). Thus, references herein to nucleic acid molecules include PNA oligomers and other nucleic acid analogs. Other examples of nucleic acid analogs and alternative/modified nucleic acid chemistries known in the art are described in Current Protocols in Nucleic Acid Chemistry, John Wiley & Sons, N.Y. (2002). Isolated nucleic acids of the inventions are comprised of base analogs including, but not limited to, any of the known base analogs of DNA and RNA such as, but not limited to 4-acetylcytosine, 8-hydroxy-N-6-methyladenosine, aziridinylcytosine, pseudoisocytosine, 5-(carboxyhydroxylmethyl)uracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methyl guanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, 2,6-diaminopurine, and 2′-modified analogs such as, but not limited to 0-methyl, amino-, and fluoro-modified analogs.

The modified or synthetic oligos and compositions of the invention are modified to enhance stability by modification with nuclease resistant groups, e.g., 2′-amino, 2′-Callyl, 2′-fluoro, 2′-0-methyl, and 2′-H. (For a review see Usman and Cedergren, TIBS 17:34, 1992; Usman, et al., Nucleic Acids Symp. Ser. 31:163, 1994). Modified oligos and compositions are purified by gel electrophoresis using general methods or can be purified by high pressure liquid chromatography and re-suspended in water.

Chemically synthesizing nucleic acid molecules with modifications (base, sugar and/or phosphate) prevents their degradation by serum ribonucleases, which increases their potency. See, e.g., Eckstein, et al., International Publication No. WO 92/07065; Perrault, et al., Nature 344:565, 1990; Pieken, et al., Science 253:314, 1991; Usman and Cedergren, Trends in Biochem. Sci. 17:334, 1992; Usman, et al, International Publication No. WO 93/15187; and Rossi, et al., International Publication No. WO 91/03162; Sproat, U.S. Pat. No. 5,334,711; Gold, et al., U.S. Pat. No. 6,300,074. All of the above references describe various chemical modifications that are made to the base, phosphate and/or sugar moieties of the isolated nucleic acid molecules described herein.

There are several examples in the art describing sugar, base and phosphate modifications that are introduced into isolated nucleic acid molecules of the invention with significant enhancement in their nuclease stability and efficacy. For example, oligonucleotides are modified to enhance stability and/or enhance biological activity by modification with nuclease resistant groups, e.g., T-amino, 2′-C-allyl, 2′-fluoro, 2′-0-methyl, 2′-H, nucleotide base modifications. For a review see Usman and Cedergren, TIBS 17:34, 1992; Usman, et al., Nucleic Acids Symp. Ser. 31:163, 1994; Burgin, et al., Biochemistry 35:14090, 1996. Sugar modification of nucleic acid molecules have been extensively described in the art. See Eckstein, et al., International Publication PCT No. WO 92/07065; Perrault, et al., Nature 344:565-568, 1990; Pieken, et al., Science 253:314-317, 1991; Usman and Cedergren, Trends in Biochem. Sci. 17:334339, 1992; Usman, et al., International Publication PCT No. WO 93/15187; Sproat, U.S. Pat. No. 5,334,711 and Beigelman, et al., J. Biol. Chem. 270:25702, 1995; Beigelman, et al., International PCT publication No. WO 97/26270; Beigelman, et al., U.S. Pat. No. 5,716,824; Usman, et al., U.S. Pat. No. 5,627,053; Woolf, et al., International PCT Publication No. WO 98/13526; Thompson, et al., Karpeisky, et al., Tetrahedron Lett. 39:1131, 1998; Earnshaw and Gait, Biopolymers (Nucleic Acid Sciences) 48:39-55, 1998; Verma and Eckstein, Annu. Rev. Biochem. 67:99-134, 1998; and Burlina, et al, Bioorg. Med. Chem. 5:1999-2010, 1997. Such publications describe general methods and strategies to determine the location of incorporation of sugar, base and/or phosphate modifications and the like into nucleic acid molecules without modulating catalysis. In view of such teachings, similar modifications are used as described herein to modify the oligonucleotide molecules of the invention so long as the ability of the modified oligos to promote gene silencing in cells is not significantly inhibited.

While chemical modification of oligonucleotide internucleotide linkages with phosphorothioate, phosphorodithioate, and/or 5′-methylphosphonate linkages improves stability, excessive modifications can cause some toxicity or decreased activity. Therefore, when engineering isolated nucleic acid molecules of the invention, the amount of these internucleotide linkages are minimized. The reduction in the concentration of these linkages lowers toxicity, resulting in increased efficacy and higher specificity of these molecules.

In one embodiment, the invention provides modified or synthetic oligo molecules, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, “Nucleic Acid Analogues: Synthesis and Properties, in Modern Synthetic Methods,” VCH, 331-417, 1995, and Mesmaeker, et al, “Novel Backbone Replacements for Oligonucleotides, in Carbohydrate Modifications in Antisense Research,” ACS, 24-39, 1994.

Further variants of the nucleic acid molecules including, but not limited to those identified as SEQ ID NOs: 14-36, such as naturally occurring allelic variants (as well as orthologs and paralogs) and synthetic variants produced by mutagenesis techniques, can be identified and/or produced using methods well known in the art. Such further variants can comprise a nucleotide sequence that shares at least 70-80%, 80-85%, 85-90%, 91%, 92%, 93%, 94%, 95%, 96%, 97%, 98%, or 99% sequence identity with a nucleic acid sequence disclosed as SEQ ID NOs: 14-36 (or a fragment thereof). Thus, the present invention specifically contemplates isolated nucleic acid molecule that have a certain degree of sequence variation compared with the sequences of SEQ ID NOs: 14-36.

The modified oligos and compositions of the invention are routinely made through techniques such as solid phase synthesis. Equipment for such synthesis is sold by several vendors including, for example, Applied Biosystems, (Foster City, Calif.). Any other means for such synthesis known in the art is additionally or alternatively employed. It is well known to use similar techniques to prepare oligonucleotides such as the phosphorothioates and alkylated derivatives.

Oligonucleotides (e.g., certain modified oligonucleotides or portions of oligonucleotides lacking ribonucleotides) are synthesized using protocols known in the art, e.g., as described in Caruthers, et al., Methods in Enzymology 211:3-19, 1992; Thompson, et al., International PCT Publication No. WO 99/54459; Wincott, et al., Nucleic Acids Res. 23:2677-2684, 1995; Wincott, et al., Methods Mol. Bio. 74:59, 1997; Brennan, et al., Biotechnol Bioeng. 61:33-45, 1998; and Brennan, U.S. Pat. No. 6,001,311. Synthesis of RNA, including certain modified oligo molecules of the invention, follows general procedures as described, e.g., in Usman, et al, J. Am. Chem. Soc. 109:7845, 1987; Scaringe, et al., Nucleic Acids Res. 18:5433, 1990; and Wincott, et al., Nucleic Acids Res. 23:2677-2684, 1995; Wincott, et al., Methods Mol. Bio. 74:59, 1997.

The comparison of sequences and determination of percent identity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In a preferred embodiment, the percent identity between two amino acid sequences is determined using the Needleman and Wunsch algorithm (J. Mol. Biol. (48):444-453 (1970)) which has been incorporated into the GAP program in the GCG software package, using either a Blossom 62 matrix or a PAM250 matrix, and a gap weight of 16, 14, 12, 10, 8, 6, or 4 and a length weight of 1, 2, 3, 4, 5, or 6.

In yet another preferred embodiment, the percent identity between two nucleotide sequences is determined using the GAP program in the GCG software package (Devereux, J., et al., Nucleic Acids Res. 12(1):387 (1984)), using a NWSgapdna.CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Myers and W. Miller (CABIOS, 4:11-17 (1989)) which has been incorporated into the ALIGN program (version 2.0), using a PAM120 weight residue table, a gap length penalty of 12, and a gap penalty of 4.

The nucleotide and amino acid sequences of the present invention can further be used as a “query sequence” to perform a search against sequence databases to, for example, identify other family members or related sequences. Such searches can be performed using the NBLAST and) (BLAST programs (version 2.0) of Altschul, et al. (J. Mol. Biol. 215:403-10 (1990)). BLAST nucleotide searches can be performed with the NBLAST program, score=100, wordlength=12 to obtain nucleotide sequences homologous to the nucleic acid molecules of the invention. BLAST protein searches can be performed with the XBLAST program, score=50, wordlength=3 to obtain amino acid sequences homologous to the proteins of the invention. To obtain gapped alignments for comparison purposes, Gapped BLAST can be utilized as described in Altschul et al. (Nucleic Acids Res. 25(17):3389-3402 (1997)). When utilizing BLAST and gapped BLAST programs, the default parameters of the respective programs (e.g.,) (BLAST and NBLAST) can be used. In addition to BLAST, examples of other search and sequence comparison programs used in the art include, but are not limited to, FASTA (Pearson, Methods Mol. Biol. 25, 365-389 (1994)) and KERR (Dufresne et al., Nat Biotechnol 2002 December; 20(12): 1269-71). For further information regarding bioinformatics techniques, see Current Protocols in Bioinformatics, John Wiley & Sons, Inc., N.Y.

### Therapeutic Methods

The nucleic acid molecules of the present invention are used to modify the expression of genes containing mutations that lead to altered wild type miRNA binding, and thus, the development of a variety of disorders. Exemplary disorders include but are not limited to, developmental, aging, inflammatory, degenerative, metabolic, proliferative, circulatory, cognitive, reproductive, and behavioral disorders. In a preferred embodiment of the invention the disorder is cancer. For example, the isolated nucleic acid molecules of the invention bind altered or mutated miRNA binding sites with a binding efficacy that equals or exceeds the binding efficacy achieved by at least one endogenous or naturally-occurring miRNA when bound to the corresponding wild type or altered/mutated miRNA binding site. In one aspect of the invention, isolated nucleic acid molecules used to modify gene expression are modified oligos.

A therapeutically effective amount of a composition of the invention is an amount of a modified oligo, or a precursor thereof, that when administered to a subject, results in the silencing, or decreased expression, of at least one gene or mRNA transcript. The effectiveness of administration of a pharmaceutical composition of the invention is measured, in this embodiment, by testing a subject, e.g. biopsied tissue or a bodily fluid, for decreased gene expression using art-recognized methods.

Alternatively, or in addition, a pharmaceutically effective amount of a composition of the invention is an amount of a modified oligo, or a precursor thereof, that prevents, inhibits the occurrence or reoccurrence of, treats, or alleviates a sign or symptom (to some extent) of a disorder. In a preferred embodiment, a pharmaceutically effective dose is that dose required to alleviate at least one sign or symptom of cancer. As used herein, the term “treat” is meant to describe a process by which a sign or symptom of a disorder, such as cancer, is eliminated. Alternatively, or in addition, a disorder such as cancer, which can occur in multiple locations, is treated if the cancer is eliminated within at least one of multiple locations.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

In one aspect of the invention, a therapeutically effective amount of a composition of the invention is an amount of a modified oligo, or a precursor thereof, that provides a preventative benefit to the subject. As used herein, the term “preventative benefit” is meant to describe a delay in the development or decrease of the severity of a sign or symptom of a disorder, such as cancer.

The pharmaceutically effective dose depends on the type of disease, the composition used, the route of administration, the individual and physical characteristics of the subject under consideration (for example, age, gender, weight, diet, smoking-habit, exercise-routine, genetic background, medical history, hydration, blood chemistry), concurrent medication, and other factors that those skilled in the medical arts will recognize.

Generally, an amount from about 0.01 mg/kg and 25 mg/kg body weight/day of active ingredients is administered dependent upon potency of the negatively charged polymer, e.g. the modified oligo composition. In alternative embodiments dosage ranges include, but are not limited to, 0.01-0.1 mg/kg, 0.01-1 mg/kg, 0.01-10 mg/kg, 0.01-20 mg/kg, 0.01-30 mg/kg, 0.01-40 mg/kg, 0.01-50 mg/kg, 0.01-60 mg/kg, 0.01-70 mg/kg, 0.01-80 mg/kg, 0.01-90 mg/kg, 0.01-100 mg/kg, 0.01-150 mg/kg, 0.01-200 mg/kg, 0.01-250 mg/kg, 0.01-300 mg/kg, 0.01-500 mg/kg, and all ranges and points in between. In alternative embodiments dosage ranges include, but are not limited to, 0.01-1 mg/kg, 1-10 mg/kg, 10-20 mg/kg, 20-30 mg/kg, 30-40 mg/kg, 40-50 mg/kg, 50-60 mg/kg, 60-70 mg/kg, 70-80 mg/kg, 80-90 mg/kg, 90-100 mg/kg, 100-150 mg/kg, 150-200 mg/kg, 200-300 mg/kg, 300-500 mg/kg, and all ranges and points in between.

The blood plasma concentration of a composition or a modified oligonucleotide can be about 0.1 μM to about 1000 μM, about 0.1 μM to about 1 μM; about 0.1 μM to about 10 μM; about 10 μM to about 100 μM; about 100 μM to about 500 μM, about 500 μM to about 1000 μM, and any micromolar concentration in between. Alternatively, or in addition, the cerebral spinal fluid concentration of a composition or a modified oligonucleotide can be about 0.1 μM to about 1000 μM, about 0.1 μM to about 1 μM; about 0.1 μM to about 10 μM; about 10 μM to about 100 μM; about 100 μM to about 500 μM, about 500 μM to about 1000 μM, or any micromolar concentration in between.

The pharmaceutical composition can be administered at a dosage from about 1 mg/m² to 5000 mg/m² per day, about 1 mg/m² to 10 mg/m² per day, about 10 mg/m² to 100 mg/m² per day, about 100 to 1000 m g/m² per day, about 1000 to 2500 mg/m² per day, about 2500 to 5000 mg/m² per day, or any daily mg/m² dosage in between. Preferably, 1 mg/m² to 5000 mg/m² per day is the administered dosage for a human.

In invention provides methods of treating or alleviating a symptom of a cell proliferative disorder. Exemplary cell proliferative disorders of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue.

In a preferred embodiment of the invention, the methods provided herein are used to treat or alleviate a symptom of cancer. Exemplary cancers include, but are not limited to, acute lymphoblastic leukemia, acute myeloid leukemia, adrenocortical carcinoma, adrenocortical carcinoma, AIDS-related cancers, AIDS-related lymphoma, anal cancer, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, islet cell tumors (endocrine pancreas), Kaposi Sarcoma, kidney (renal cell) cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, non-small cell lung cancer, small cell lung cancer, AIDS-related lymphoma, non-Hodgkin lymphoma, primary central nervous system lymphoma, Waldenstram macroglobulinemia, medulloblastoma, melanoma, intraocular (eye) melanoma, merkel cell carcinoma, mesothelioma malignant, mesothelioma, metastatic squamous neck cancer, mouth cancer, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, uterine sarcoma, skin cancer (nonmelanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilms Tumor.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

But sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage.

A cancer may also cause symptoms such as fever, fatigue, or weight loss. This may be because cancer cells use up much of the body's energy supply or release substances that change the body's metabolism. Or the cancer may cause the immune system to react in ways that produce these symptoms.

Sometimes, cancer cells release substances into the bloodstream that cause symptoms not usually thought to result from cancers. For example, some cancers of the pancreas can release substances which cause blood clots to develop in veins of the legs. Some lung cancers make hormone-like substances that affect blood calcium levels, affecting nerves and muscles and causing weakness and dizziness.

Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma.

Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease.

Along with cancers of the skin (see next section), some internal cancers can cause skin signs that can be seen. These changes include the skin looking darker (hyperpigmentation), yellow (jaundice), or red (erythema); itching; or excessive hair growth.

Alternatively, or in addition, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could indicate cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer.

Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size.

Indigestion or trouble swallowing could indicate cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma.

A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant invention.

The methods of the invention encompass a variety of subjects, all of whom are mammals. In certain embodiments, the mammal is a human, non-human primate, mouse, rat, dog, cat, horse, or cow, but are not limited to these examples. Mammals other than humans are advantageously used as subjects that represent animal models of a particular disorder. The preferred subject is human. A subject is male or female.

Subjects are identified as having a mutation in a miRNA binding site. Subjects having been identified with at least one mutation in a miRNA binding site, have not presented signs or symptoms of a disorder, such as cancer. Alternatively, subjects having been identified with at least one mutation in a miRNA binding site, have presented signs or symptoms of a disorder, such as cancer. Optionally, subjects have been diagnosed with one or more disorders, such as cancer. In certain embodiments of the invention, subjects have been diagnosed with cancers most frequently associated with the presence of the LCS6 SNP including, but not limited to, all varieties of lung cancer (e.g., non-small cell lung cancer (NSCLC) and small cell lung cancer), ovarian cancer, breast cancer, uterine cancer, head and neck cancer, pancreatic cancer, and colon cancer.

Subjects of the methods herein may not have any mutations in a miRNA binding site. Many of the genes that are overexpressed in cancer contain let-7 miRNA binding sites. As such, the modified oligos and compositions of the invention, when administered to a subject lacking mutations, will repress such overexpression of let-7 target genes. In certain embodiments, the gene expression profiles of intended subjects are analyzed and the appropriate modified oligos capable of silencing gene overexpression are administered. For example, a modified oligo (SEQ ID NOs: 22-36) that binds the LCS6 SNP containing KRAS allele, also binds a wild type KRAS allele and effectively decreases expression.

The invention provides a method of treating or alleviating a symptom of cancer by administering a composition comprising a modified oligo and at least one cytotoxic compound. Cytotoxic compounds include, but are not limited to, all forms of radioactive isotopes and chemotherapeutic compounds, e.g. chemotherapy drugs. Exemplary radioactive isotopes include, but are not limited to, molybdenum-99, technetium-99m, bismuth-213, carbon-11, chromium-51, cobalt-57, cobalt-60, copper-64, dysprosium-165, erbium-169, fluorine-18, gallium-67, holmium-166, indium-111, iodine-123, iodine-125, iodine-131, iridium-192, iron-59, krypton-81m, lutetium-177, nitrogen-13, oxygen-15, palladium-103, phosphorus-32, potassium-42, rhenium-186, rhenium-188, rubidium-81, samarium-153, selenium-75, sodium-24, strontium-89, strontium-92, thallium-201, xenon-133, ytterbium-177, ytterbium-169, yttrium-90, and radioisotopes of caesium, gold, and rthenium. Exemplary chemotherapy drugs include, but are not limited to, Dacarbazine/DTIC, Fluorouracil/5-FU, Fludarabine, Gemcitabine, Trastuzumab/Herceptin, Hydroxyurea/Hydrea, Idarubicin, Ifosfamide, Irinotecan, Cladribine/Leustatin, Mercaptopurine/Purinethol/6-MP, Methotrexate, Mithramycin/Plicamycin, Mitomycin, Mitoxanthrone/Novatrone, Navelbine/Vinorelbine, Nitrogen Mustard, Rituxan, Paclitaxel/Taxol, Docetaxel/Taxotere, Topotecan, Velban/Vinblastine, Vincristine, and Etoposide/VP-16.

### Pharmaceutical Compositions

The invention provides a composition including at least one modified oligo and a pharmaceutically acceptable carrier. Pharmaceutically acceptable carriers are covalently or non-covalently bound, admixed, encapsulated, conjugated, operably-linked, or otherwise associated with the modified oligo such that the pharmaceutically acceptable carrier increases the cellular uptake, stability, solubility, half-life, binding efficacy, specificity, targeting, distribution, absorption, or renal clearance of the modified oligo molecule. Alternatively, or in addition, the pharmaceutically acceptable carrier increases or decreases the immunogenicity of the modified oligo molecule. Furthermore, the pharmaceutically acceptable carrier is capable to increasing the cytotoxicity of the modified oligo composition with respect to the targeted cancer cells.

Alternatively, or in addition, pharmaceutically acceptable carriers are salts (for example, acid addition salts, e.g., salts of hydrochloric, hydrobromic, acetic acid, and benzene sulfonic acid), esters, salts of such esters, or any other compound which, upon administration to a subject, are capable of providing (directly or indirectly) the biologically active compositions of the invention. As such, the invention encompasses prodrugs, and other bioequivalents. As used herein, the term “prodrug” is meant to describe, a pharmacological substance that is administered in an inactive (or significantly less active) form. Once administered, the prodrug is metabolized in vivo into an active metabolite. Pharmaceutically acceptable carriers are alternatively or additionally diluents, excipients, adjuvants, emulsifiers, buffers, stabilizers, and/or preservatives.

Pharmaceutically acceptable carriers of the invention are modified oligo delivery systems/mechanisms that increase uptake of the modified oligo by targeted cells. For example, pharmaceutically acceptable carriers of the invention are viruses, recombinant viruses, engineered viruses, viral particles, replication-deficient viruses, liposomes, cationic lipids, anionic lipids, cationic polymers, polymers, hydrogels, micro- or nano-capsules (biodegradable), micropheres (optionally bioadhesive), cyclodextrins, plasmids, mammalian expression vectors, proteinaceous vectors, or any combination of the preceding elements (see, O'Hare and Normand, International PCT Publication No. WO 00/53722; U.S. Patent Publication 2008/0076701). Moreover, pharmaceutically acceptable carriers that increase cellular uptake can be modified with cell-specific proteins or other elements such as receptors, ligands, antibodies to specifically target cellular uptake to a chosen cell type.

In another aspect of the invention, compositions are first introduced into a cell or cell population that is subsequently administered to a subject. In some embodiments, a modified oligo is delivered intracellularly, e.g., in cells of a target tissue such as lung, or in inflamed tissues. Included within the invention are compositions and methods for delivery of an isolated modified oligo and/or composition by removing cells of a subject, delivering the isolated modified oligo or composition to the removed cells, and reintroducing the cells into a subject. In some embodiments, a modified oligo molecule is combined with a cationic lipid or transfection material such as LIPOFECTAMINE (Invitrogen).

In one aspect, the active compounds are prepared with pharmaceutically acceptable carriers that will protect the modified oligo molecule against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Examples of materials which can form hydrogels include polylactic acid, polyglycolic acid, PLGA polymers, alginates and alginate derivatives, gelatin, collagen, agarose, natural and synthetic polysaccharides, polyamino acids such as polypeptides particularly poly(lysine), polyesters such as polyhydroxybutyrate and poly-epsilon.-caprolactone, polyanhydrides; polyphosphazines, poly(vinyl alcohols), poly(alkylene oxides) particularly poly(ethylene oxides), poly(allylamines) (PAM), poly(acrylates), modified styrene polymers such as poly(4-aminomethylstyrene), pluronic polyols, polyoxamers, poly(uronic acids), poly(vinylpyrrolidone) and copolymers of the above, including graft copolymers.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Pharmaceutically acceptable carriers are cationic lipids that are bound or associated with modified oligos. Alternatively, or in addition, modified oligos are encapsulated or surrounded in cationic lipids, e.g. lipsosomes, for in vivo delivery. Exemplary cationic lipids include, but are not limited to, N-41-(2,3-dioleoyloxy)propyli-N,N,N-trimethylammonium chloride (DOTMA); 1,2-bis(oleoyloxy)-3-3-(trimethylammonium)propane (DOTAP), 1,2-bis(dimyrstoyloxy)-3-3-(trimethylammonia)propane (DMTAP); 1,2-dimyristyloxypropyl-3-dimethylhydroxyethylammonium bromide (DMRIE); dimethyldioctadecylammonium bromide (DDAB); 3-(N—(N′,N′-dimethylaminoethane)carbamoyl)cholesterol (DC-Chol); 3.beta.-[N′,N′-diguanidinoethyl-aminoethane)carbamoyl cholesterol (BGTC); 2-(2-(3-(bis(3-aminopropyl)amino)propylamino)acetamido)-N,N-ditetradecyla-cetamide (RPR209120); pharmaceutically acceptable salts thereof, and mixtures thereof. Further examplary cationic lipids include, but are not limited to, 1,2-dialkenoyl-sn-glycero-3-ethylphosphocholines (EPCs), such as 1,2-dioleoyl-sn-glycero-3-ethylphosphocholine, 1,2-distearoyl-sn-glycero-3-ethylphosphocholine, 1,2-dipalmitoyl-sn-glycero-3-ethylphosphocholine, pharmaceutically acceptable salts thereof, and mixtures thereof.

Exemplary polycationic lipids include, but are not limited to, tetramethyltetrapalmitoyl spermine (TMTPS), tetramethyltetraoleyl spermine (TMTOS), tetramethlytetralauryl spermine (TMTLS), tetramethyltetramyristyl spermine (TMTMS), tetramethyldioleyl spermine (TMDOS), pharmaceutically acceptable salts thereof, and mixtures thereof. Further examplary polycationic lipids include, but are not limited to, 2,5-bis(3-aminopropylamino)-N-(2-(dioctadecylamino)-2-oxoethyl)pentanamid-e (DOGS); 2,5-bis(3-aminopropylamino)-N-(2-(di(Z)-octadeca-9-dienylamino)--2-oxoethyl)pentanamide (DOGS-9-en); 2,5-bis(3-aminopropylamino)-N-(2-(di(9Z,12Z)-octadeca-9,12-dienylamino)-2--oxoethyl)pentanamide (DLinGS); 3-beta-(N.sup.4-(N.sup.1, N.sup.8-dicarbobenzoxyspermidine)carbamoyl)chole-sterol (GL-67); (9Z,9^{y}Z)-2-(2,5-bis(3-aminopropylamino)pentanamido)propane-1,3-diyl-dioct-adec-9-enoate (DOSPER); 2,3-dioleyloxy-N-[2(sperminecarboxamido)ethyl]-N,N-dimethyl-1-propanamini-urn trifluoro-acetate (DOSPA); pharmaceutically acceptable salts thereof, and mixtures thereof.

Examples of cationic lipids are described in U.S. Pat. Nos. 4,897,355; 5,279,833; 6,733,777; 6,376,248; 5,736,392; 5,334,761; 5,459,127; 2005/0064595; U.S. Pat. Nos. 5,208,036; 5,264,618; 5,279,833; 5,283,185; 5,753,613; and 5,785,992; each of which is incorporated herein in its entirety.

Pharmaceutically acceptable carriers of the invention also include non-cationic lipids, such as neutral, zwitterionic, and anionic lipids. Examplary non-cationic lipids include, but are not limited to, 1,2-Dilauroyl-sn-glycerol (DLG); 1,2-Dimyristoyl-sn-glycerol (DMG); 1,2-Dipalmitoyl-sn-glycerol (DPG); 1,2-Distearoyl-sn-glycerol (DSG); 1,2-Dilauroyl-sn-glycero-3-phosphatidic acid (sodium salt; DLPA); 1,2-Dimyristoyl-snglycero-3-phosphatidic acid (sodium salt; DMPA); 1,2-Dipalmitoyl-sn-glycero-3-phosphatidic acid (sodium salt; DPPA); 1,2-Distearoyl-sn-glycero-3-phosphatidic acid (sodium salt; DSPA); 1,2-Diarachidoyl-sn-glycero-3-phosphocholine (DAPC); 1,2-Dilauroyl-sn-glycero-3-phosphocholine (DLPC); 1,2-Dimyristoyl-sn-glycero-3-phosphocholine (DMPC); 1,2-Dipalmitoyl-sn-glycero-0-ethyl-3-phosphocholine (chloride or triflate; DPePC); 1,2-Dipalmitoyl-sn-glycero-3-phosphocholine (DPPC); 1,2-Distearoyl-sn-glycero-3-phosphocholine (DSPC); 1,2-Dilauroyl-sn-glycero-3-phosphoethanolamine (DLPE); 1,2-Dimyristoyl-sn-glycero-3-phosphoethanolamine (DMPE); 1,2-Dipalmitoyl-sn-glycero-3-phosphoethanolamine (DPPE); 1,2-Distearoylsn-glycero-3-phosphoethanolamine (DSPE); 1,2-Dilauroyl-sn-glycero-3-phosphoglycerol (sodium salt; DLPG); 1,2-Dimyristoyl-sn-glycero-3-phosphoglycerol (sodium salt; DMPG); 1,2-Dimyristoyl-sn-glycero-3-phospho-sn-1-glycerol (ammonium salt; DMP-sn1-G); 1,2-Dipalmitoyl-sn-glycero-3-phosphoglycerol (sodium salt; DPPG); 1,2-Distearoyl-sn-glycero-3-phosphoglycero (sodium salt; DSPG); 1,2-Distearoyl-snglycero-3-phospho-sn-1-glycerol (sodium salt; DSP-sn-1-G); 1,2-Dipalmitoyl-snglycero-3-phospho-L-serine (sodium salt; DPP S); 1-Palmitoyl-2-linoleoyl-sn-glycero-3-phosphocholine (PLinoPC); 1-Palmitoyl-2-oleoyl-sn-glycero-3-phosphocholine (POPC); 1-Palmitoyl-2-oleoyl-sn-glycero-3-phosphoglycerol (sodium salt; POPG); 1-Palmitoyl-2-oleoyl-sn-glycero-3-phosphoglycerol (sodium salt; POPG); 1-Palmitoyl-2-oleoyl-snglycero-3-phosphoglycerol (ammonium salt; POPG); 1-Palmitoyl-2-4o-sn-glycero-3-phosphocholine (P-lyso-PC); 1-Stearoyl-2-lyso-sn-glycero-3-phosphocholine (S-lysoPC); and mixtures thereof. Further examplary non-cationic lipids include, but are not limited to, polymeric compounds and polymer-lipid conjugates or polymeric lipids, such as pegylated lipids, including polyethyleneglycols, N-(Carbonyl-methoxypolyethyleneglycol-2000)-1,2-dimyristoyl-sn-glycero-3-phosphoethanolamine (sodium salt; DMPE-MPEG-2000); N-(Carbonyl-methoxypolyethyleneglycol-5000)-1,2-dimyristoyl-sn-glycero-3-phosphoethanolamine (sodium salt; DMPE-MPEG-5000); N-(Carbonyl-methoxypolyethyleneglycol 2000)-1,2-dipalmitoyl-sn-glycero-3-phosphoethanolamine (sodium salt; DPPE-MPEG-2000); N-(Carbonyl-methoxypolyethyleneglycol 5000)-1,2-dipalmitoyl-sn-glycero-3-phosphoethanolamine (sodium salt; DPPE-MPEG-5000); N-(Carbonyl-methoxypolyethyleneglycol 750)-1,2-distearoyl-sn-glycero-3-phosphoethanolamine (sodium salt; DSPE-MPEG-750); N-(Carbonyl-methoxypolyethyleneglycol 2000)-1,2-distearoyl-sn-glycero-3-phosphoethanolamine (sodium salt; DSPE-MPEG-2000); N-(Carbonyl-methoxypolyethyleneglycol 5000)-1,2-distearoyl-sn-glycero-3-phosphoethanolamine (sodium salt; DSPE-MPEG-5000); sodium cholesteryl sulfate (SCS); pharmaceutically acceptable salts thereof, and mixtures thereof. Examples of non-cationic lipids include, but are not limited to, dioleoylphosphatidylethanolamine (DOPE), diphytanoylphosphatidylethanolamine (DPhPE),1,2-Dioleoyl-sn-Glycero-3-Phosphocholine (DOPC), 1,2-Diphytanoyl-sn-Glycero-3-Phosphocholine (DPhPC), cholesterol, and mixtures thereof.

Pharmaceutically-acceptable carriers of the invention further include anionic lipids. Examplary anionic lipids include, but are not limited to, phosphatidylserine, phosphatidic acid, phosphatidylcholine, platelet-activation factor (PAF), phosphatidylethanolamine, phosphatidyl-DL-glycerol, phosphatidylinositol, phosphatidylinositol (pi(4)p, pi(4,5)p2), cardiolipin (sodium salt), lysophosphatides, hydrogenated phospholipids, sphingoplipids, gangliosides, phytosphingosine, sphinganines, pharmaceutically acceptable salts thereof, and mixtures thereof.

Supplemental or complementary methods for delivery of nucleic acid molecules for use herein are described, e.g., in Akhtar, et al., Trends Cell Bio. 2:139, 1992; Delivery Strategies for Antisense Oligonucleotide Therapeutics, ed. Akhtar, 1995; Maurer, et al., Mol. Membr. Biol. 16:129-140, 1999; Hofland and Huang, Handb. Exp. Pharmacol. 137:165-192, 1999; and Lee, et al., ACS Symp. Ser. 752:184-192, 2000. Sullivan, et al., International PCT Publication No. WO 94/02595, further describes general methods for delivery of enzymatic nucleic acid molecules. These protocols can be utilized to supplement or complement delivery of virtually any nucleic acid molecule of the invention.

Pharmaceutical compositions are administered locally and/or systemically. As used herein, the term “local administration” is meant to describe the administration of a pharmaceutical composition of the invention to a specific tissue or area of the body with minimal dissemination of the composition to surrounding tissues or areas. Locally administered pharmaceutical compositions are not detectable in the general blood stream when sampled at a site not immediate adjacent or subjacent to the site of administration.

As used herein the term “systemic administration” is meant to describe in vivo systemic absorption or accumulation of drugs in the blood stream followed by distribution throughout the entire body. Administration routes which lead to systemic absorption include, without limitation: intravenous, subcutaneous, intraperitoneal, inhalation, oral, intrapulmonary and intramuscular. Each of these administration routes exposes the desired negatively charged polymers, e.g., nucleic acids, to an accessible diseased tissue. The rate of entry of a drug into the circulation has been shown to be a function of molecular weight or size. The use of a liposome or other drug carrier comprising the compounds of the instant disclosure can potentially localize the drug, e.g., in certain tissue types, such as the tissues of the reticular endothelial system (RES). A liposome formulation that can facilitate the association of drug with the surface of cells, such as, lymphocytes and macrophages is also useful. This approach may provide enhanced delivery of the drug to target cells by taking advantage of the specificity of macrophage and lymphocyte immune recognition of abnormal cells, such as cancer cells.

A pharmaceutically acceptable carrier is chosen to be compatible with its intended route of administration. Examples of routes of administration include parenteral, e.g., intravenous, intradermal, subcutaneous, oral (e.g., inhalation or insufflation), transdermal (topical), transmucosal, transopthalmic, tracheal, intranasal, epidermal, intraperitoneal, intraorbital, intraarterial, intracapsular, intraspinal, intrasternal, intracranial, intrathecal, intraventricular, and rectal administration. Alternatively, or in addition, compositions of the invention are administered non-parentally, for example, orally. Alternatively, or further in addition, compositions of the invention are administered surgically, for example, as implants or biocompatible polymers.

Pharmaceutical compositions are administered via injection or infusion, e.g. by use of an infusion pump. Direct injection of the nucleic acid molecules of the invention, is performed using standard needle and syringe methodologies, or by needle-free technologies such as those described in Conry et al., Clin. Cancer Res. 5:2330-2337, 1999 and Barry et al., International PCT Publication No. WO 99/31262.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

An isolated nucleic acid with a pharmaceutically acceptable carrier of the invention can be administered to a subject in many of the well-known methods currently used for chemotherapeutic treatment. For example, for treatment of cancers, a compound of the invention may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the subject should preferably be closely monitored during and for a reasonable period after treatment.

Compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof.

The pharmaceutical compositions are in the form of a sterile injectable aqueous or oleaginous suspension. This suspension is formulated according to the known art using those suitable dispersing or wetting agents and suspending agents that have been mentioned above. The sterile injectable preparation is a sterile injectable solution or suspension in a non-toxic parentally acceptable diluent or solvent, e.g., as a solution in 1,3-butanediol. Exemplary acceptable vehicles and solvents are water, Ringer's solution and isotonic sodium chloride solution. In addition, sterile, fixed oils are conventionally employed as a solvent or suspending medium. For this purpose, any bland fixed oil is employed including synthetic mono- or diglycerides. In addition, fatty acids such as oleic acid are used in the preparation of injectables.

Sterile injectable solutions can be prepared by incorporating the modified oligo in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. Modified oligos containing at least one 2′-0-methoxyethyl modification are used when formulating compositions for oral administration. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Exemplary penetrants for transdermal administration include, but are not limited to, lipids, liposomes, fatty acids, fatty acid, esters, steroids, chelating agents, and surfactants. Preferred lipids and liposomes of the invention are neutral, negative, or cationic. Compositions are encapsulated within liposomes or form complexes thereto, such as cationic liposomes.

Alternatively, or in addition, compositions are complexed to lipids, such as cationic lipids. Compositions prepared for transdermal administration are provided by iontophoresis. Such penetrants are generally known in the art, and include, for example, for transmucosal administration, detergents, bile salts, and fusidic acid derivatives.

Transmucosal administration can be accomplished through the use of nasal sprays or suppositories. For transdermal administration, the active compounds are formulated into patches, ointments, lotions, salves, gels, drops, sprays, liquids, powders, or creams as generally known in the art.

Pharmaceutical compositions of the invention are administered systemically and are intended to cross the blood-brain barrier to contact cells of the central nervous system. Alternatively, or in addition, pharmaceutical compositions are administered intraspinally by, for example, lumbar puncture, or intracranially, e.g. intrathecally or intraventricularly. By the preceding routes, pharmaceutical compositions are introduced directly into the cerebral spinal fluid. Nonlimiting examples of agents suitable for formulation with the nucleic acid molecules of the invention, particularly for targeting nervous system tissues, include: P-glycoprotein inhibitors (such as Pluronic P85), which can enhance entry of drugs into the CNS (Jolliet-Riant and Tillement, Fundam. Clin. Pharmacol. 13:16-26, 1999); biodegradable polymers, such as poly (DL-lactide-coglycolide) microspheres for sustained release delivery after intracerebral implantation (Emerich, D. F., et al., Cell Transplant 8:47-58, 1999) (Alkermes, Inc. Cambridge, Mass.); and loaded nanoparticles, such as those made of polybutylcyanoacrylate, which can deliver drugs across the blood brain barrier and can alter neuronal uptake mechanisms (Prog. Neuropsychopharmacol Biol. Psychiatry 23:941-949, 1999). Other non-limiting examples of delivery strategies for the nucleic acid molecules of the instant disclosure include material described in Boado, et al., J. Pharm. Sci. 87:1308-1315, 1998; Tyler, et al., FEBS Lett. 421:280-284, 1999; Pardridge, et al, PNAS USA. 92:5592-5596, 1995; Boado, Adv. Drug Delivery Rev. 15:73-107, 1995; Aldrian-Herrada, et al., Nucleic Acids Res. 26:4910-4916, 1998; and Tyler, et al., PNAS USA. 96:7053-7058, 1999.

The modified oligos and compositions of the invention are also administered in the form of suppositories, e.g., for rectal administration of the drug. These compositions are prepared by mixing the drug with a suitable non-irritating excipient that is solid at ordinary temperatures but liquid at the rectal temperature and will therefore melt in the rectum to release the drug. Such materials include cocoa butter and polyethylene glycols.

Aqueous suspensions contain the active materials in admixture with excipients suitable for the manufacture of aqueous suspensions. Such excipients are suspending agents, e.g., sodium carboxymethylcellulose, methylcellulose, hydropropylmethylcellulose, sodium alginate, polyvinylpyrrolidone, gum tragacanth and gum acacia; dispersing or wetting agents can be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, e.g., polyethylene sorbitan monooleate. The aqueous suspensions also contain one or more preservatives, e.g., ethyl, or n-propyl phydroxybenzoate, one or more coloring agents, one or more flavoring agents, and one or more sweetening agents, such as sucrose or saccharin.

Oily suspensions are formulated by suspending the active ingredients in a vegetable oil, e.g., arachis oil, olive oil, sesame oil or coconut oil, or in a mineral oil such as liquid paraffin. The oily suspensions contain a thickening agent, e.g., beeswax, hard paraffin or cetyl alcohol. Sweetening agents and flavoring agents are added to provide palatable oral preparations. These compositions are preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, e.g., sweetening, flavoring and coloring agents, are also present.

Pharmaceutical compositions of the invention are in the form of oil-in-water emulsions. The oily phase is a vegetable oil or a mineral oil or mixtures of these. Suitable emulsifying agents are naturally-occurring gums, e.g., gum acacia or gum tragacanth, naturally-occurring phosphatides, e.g., soy bean, lecithin, and esters or partial esters derived from fatty acids and hexitol, anhydrides, e.g., sorbitan monooleate, and condensation products of the said partial esters with ethylene oxide, e.g., polyoxyethylene sorbitan monooleate. The emulsions also contain sweetening and flavoring agents.

In a preferred aspect, the pharmaceutically acceptable carrier can be a solubilizing carrier molecule. More preferably, the solubilizing carrier molecule can be Poloxamer, Povidone K17, Povidone K12, Tween 80, ethanol, Cremophor/ethanol, Lipiodol, polyethylene glycol (PEG) 400, propylene glycol, Trappsol, alpha-cyclodextrin or analogs thereof, beta-cyclodextrin or analogs thereof, and gamma-cyclodextrin or analogs thereof.

The invention also provides compositions prepared for storage or administration. Acceptable carriers or diluents for therapeutic use are well known in the pharmaceutical art, and are described, e.g., in Remington's Pharmaceutical Sciences, Mack Publishing Co., A. R. Gennaro Ed., 1985. For example, preservatives, stabilizers, dyes and flavoring agents are provided. These include sodium benzoate, sorbic acid and esters of phydroxybenzoic acid. In addition, antioxidants and suspending agents are used.

### EXAMPLES

### Example 1

### Clonogenic Assays to Measure Targeting SNPs

Cancer cells and/or cell lines of interest are transfected with targeted and control oligonucleotide, double-stranded RNA (dsRNA), and DNA molecules designed to target an identified single nucleotide polymorphism (SNP). Experiments involving cancer cells and/or cell lines that carry a mutation, often a SNP, with identical or similar cancer cells and/or cell lines that do not carry the mutation are often conducted in parallel.

The transfection method used was optimized by using a luciferase (luc) reporter construct sensitive to microRNA levels (luc fused to the NRAS 3′UTR, see NCBI Accession No. NM_{—}002524 for NRAS sequence, herein incorporated by reference). The chosen transfection method for these studies causes the least toxicity and leads to the most efficient transfection (X-tremeGENE, Roche, data not shown). Following transfection, cells are plated at a range of dilutions and grown without being disturbed for 2 weeks in order to allow for colony formation. Colony counts are performed by using cell staining procedures. The appearance of a colony represents the survival of a cell and it's clonal progeny. As such, the total amount of cell survival can be represented by the number of colonies formed as a result of each treatment.

In cases where targeting of the SNP by a nucleic acid is tested in combination with cytotoxic treatment (such as radiation or chemotherapy), cells are treated 24 hours post-transfection with increasing doses of cytotoxic treatment and then plated at a range of dilutions and grown without being disturbed. Cell survival is assessed as described supra. Experiments are performed in quadruplicate for each dose and for each SNP targeting method. Experiments are repeated at a minimum of two times. Stratified t-tests are performed to analyze statistical significance for experiments.

### Example 2

### Direct Targeting of Modified Oligos to the KRAS 3′UTR: Design of RNA-DNA Chimeras

Modified oligos, also referred to herein as siRNAs, consisting of, consisting essentially of, or comprising double stranded RNA-DNA oligomer chimeras were designed to target the LCS6 SNP. The use of RNA-DNA chimeras, as opposed to double stranded RNA-RNA chimeras, minimizes off-targeting for a number of reasons including, but not limited to, inhibiting introduction of the passenger strand (PS) into the RNA-Induced Silencing Complex (RISC), rendering the PS non-functional, and making the seed binding more specific.

The use of RNA-DNA chimeras inhibits introduction of the PS into the RISC through altered binding energies between the two strands. RNA binding to DNA (and DNA binding to DNA) shows a lower binding energy than RNA binding to RNA. The strand with the weaker binding at the 5′ end is incorporated into the RISC, therefore introducing DNA (or mismatches) at the 5′ end of the guide strand (GS) introduces a bias for incorporating the guide strand over the passenger strand. A bias for incorporation of the GS over the PS not only prevents off-targeting but also makes the siRNA more efficient.

The use of RNA-DNA chimeras renders the PS non-functional. SiRNAs with DNA bases at the 3′ end are non-functional. Thus introducing DNA bases at the 3′ end of the PS avoids off-target effects.

The use of RNA-DNA chimeras makes the seed binding more specific. As a result of the lower binding energy between DNA and RNA, siRNAs with DNA bases in the seed region (typically the first 2-10 nucleotides of the 5′ end) show higher sensitivity to mismatches in the seed region when binding to target mRNA. Thus, siRNAs with DNA bases incorporated into the seed region decrease or minimize off-targeting.

Alternatively, or in addition, modified bases like 2′OM are added into the seed region in order to further reduce off-targeting.

### Example 3

### Direct Targeting of Modified Oligos to the KRAS 3′UTR: Generation of Modified Oligo 1

Methods of the invention include directly targeting modified oligo molecules to let-7 complementary sites (LCSs) with the 3′UTRs of genes such as KRAS.

In one embodiment of the invention the targeted gene is human KRAS (known by gene ID Hs KRAS, and GenBank Accession No. M54968, herein incorporated by reference). Within this human KRAS gene, the LCS6 SNP occurs at nucleotide base pair 2509. Modified oligos of the invention not only target miRNA binding sites, but also any region or sequence that includes, contains, or comprises the LCS6 SNP. Modified oligos of the invention, for example, those provided herein are nonlimiting exemplary modified oligos. All modified oligos that specifically target a region or sequence that includes, contains, or comprises the LCS6 SNP are encompassed by the instant invention.

The following sequences relate to modified oligo 1 wherein RNA are in black small letters, DNA are in capital letters, and sequence modifications in bold. Guide Strands (GS) are paired with Passenger Strands (PS) to form double-stranded siRNA molecules. Degrees are Celcius. The “G” within the targeted sequence represents the LCS6 SNP.

| | |
|---|---|
| Targeted Sequence | cctgacctcaagtgatGcacc |
| | (SEQ ID NO: 23) |
| GS1 | TGTGCATCacuugaggucagg |
| | (SEQ ID NO: 24) |
| GS2 | ugugcaucacuugaggucagg |
| | (SEQ ID NO: 25) |
| GS3 | ggugcaucacuugaggucagg |
| | (SEQ ID NO: 26) |
| PS | UgaccucaagugaTGCACCCA |
| | (SEQ ID NO: 27) |

Melting temperatures 8 nucleotides 5′ GS:

### RNA-RNA->36.8 deg

### RNA-DNA->20.8 deg

### DNA-DNA->21.5 deg

Melting temperature 8 nucleotides 5′ PS:

### RNA-RNA->36.9 deg

Melting temperature 8 nucleotides 5′ GS with mismatch:

### RNA-DNA-><10 deg

### DNA-DNA->10.9 deg

### Example 4

### Direct Targeting of Modified Oligos to the KRAS 3′UTR: Generation of Modified Oligo 2

The following sequences relate to modified oligo 2 wherein RNA are in black small letters, DNA are in capital letters, and sequence modifications in bold. Guide Strands (GS) are paired with Passenger Strands (PS) to form double-stranded siRNA molecules. Degrees are Celsius. The “G” within the targeted sequence represents the LCS6 SNP.

| | |
|---|---|
| Targeted Sequence | ctcctgacctcaagtgatGca |
| | (SEQ ID NO: 28) |
| GS1 | ugcaucacuugaggucaggag |
| | (SEQ ID NO: 29) |
| GS2 | TGCATCACuugaggucaggag |
| | (SEQ ID NO: 30) |
| PS1 | ccugaccucaagugaugcacc |
| | (SEQ ID NO: 31) |
| PS2 | ccugaccucaaguGATGCACC |
| | (SEQ ID NO: 32) |

Melting temperature 8 nucleotides 5′ GS:

### RNA-RNA->32.0 deg

### RNA-DNA->12.5 deg

### DNA-DNA->18.4 deg

Melting temperature 8 nucleotides 5′ PS:

### RNA-RNA->40.7 deg

### Example 5

### Direct Targeting of Modified Oligos to the KRAS 3′UTR: Generation of Modified Oligo 3

The following sequences relate to modified oligo 3 wherein RNA are in black small letters, DNA are in capital letters, and sequence modifications in bold. Guide Strands (GS) are paired with Passenger Strands (PS) to form double-stranded siRNA molecules. Degrees are Celsius. The “G” within the targeted sequence represents the LCS6 SNP.

| | |
|---|---|
| Targeted sequence | actcctgacctcaagtgatGc |
| | (SEQ ID NO: 33) |
| GS | ucaucacuugaggucaggagu |
| | (SEQ ID NO: 34) |
| PS1 | uccugaccucaagTGATGCAC |
| | (SEQ ID NO: 35) |
| PS2 | uccugaccucaagugaugcac |
| | (SEQ ID NO: 36) |

Melting temperature 8 nucleotides 5′ GS:

### RNA-RNA->16.3 deg

Melting temperature 8 nucleotides 5′ PS:

### RNA-RNA->41.5 deg

### Example 6

### Direct Targeting of Modified Oligos to the KRAS 3′UTR: Luciferase Assay

The modified oligos described in Examples 3-5 were resuspended and then annealed (combinations are detailed below).

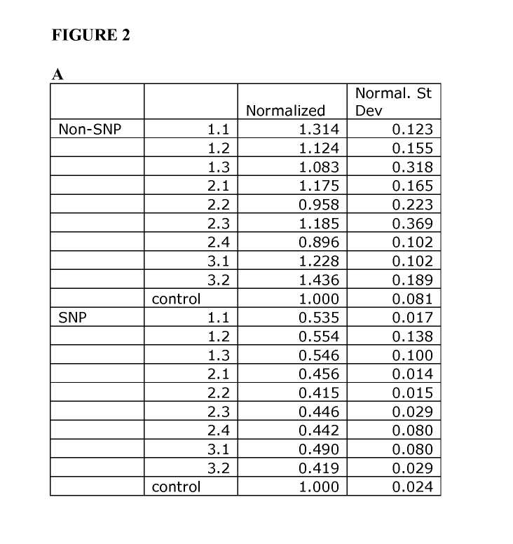

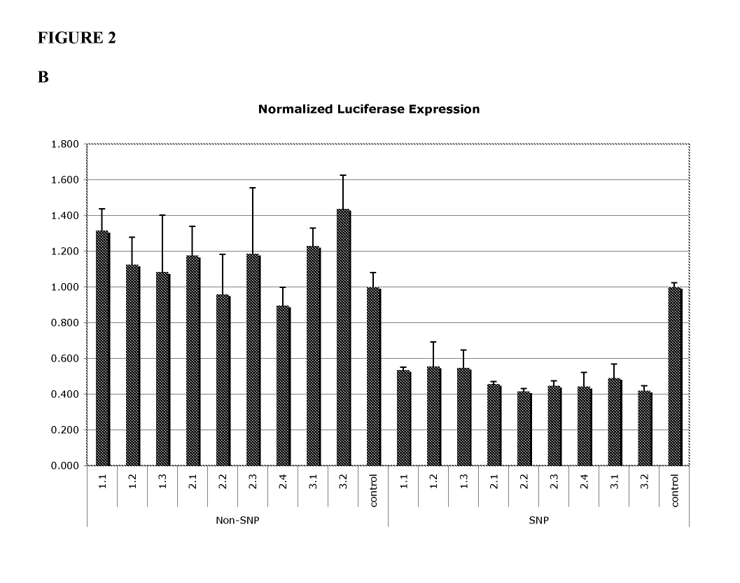

Combination of Annealed Oligos Used (see, FIGS. 2B, 3, 4, and 5):

### KRAS #1-1: KRAS1 GS1+KRAS1 PS

### KRAS #1-2: KRAS1 GS2+KRAS1 PS

### KRAS #1-3: KRAS1 GS3+KRAS1 PS

### KRAS #2-1: KRAS2 GS1+KRAS2 PS1

### KRAS #2-2: KRAS2 GS1+KRAS2 PS2

### KRAS #2-3: KRAS2 GS2+KRAS2 PS1

### KRAS #2-4: KRAS2 GS2+KRAS2 PS2

### KRAS #3-1: KRAS3 GS1+KRAS3 PS1

### KRAS #3-2: KRAS3 GS1+KRAS3 PS2

Annealed modified oligos were transfected into 293T cells using Lipofectamine™. Luciferase reporter constructs containing the KRAS 3′UTR, either with or without the LCS6 SNP, were also transfected into these 293T cells Annealed modified oligos bind to either luciferase construct and silence gene expression by either degradation of the reporter protein construct or inhibition of translation of the reporter protein construct.

The negative control used for these luciferase assays was Qiagen AllStars negative Control siRNA with no modifications. This negative control has been checked for off-targeting by microarray and has been experimentally validated for having no effect on cell cycle, viability, and/or nucleus size.

Firefly luciferase values were normalized to renilla luciferase values. The average of this ratio for the three independent transfections was calculated at each time point for each experiment. This calculated average was then normalized to the average ratio in the control siRNA transfection (FIGS. 2A and B).

Results of the luciferase reporter assay show that the annealed modified oligos directly bind the KRAS 3′UTR containing the LCS6 SNP. Moreover, the annealed modified oligos are capable of specifically silencing the reporter construct containing the LCS6 SNP demonstrated by decreased luciferase expression from this construct compared to the wild type KRAS construct (FIG. 2B). This assay is a proof-of-concept result showing that modified oligos can be engineered which directly target regions containing at least one SNP, e.g. the LCS6 SNP, and result in the silencing of the gene which contains this region.

### Example 7

### Modified Oligo Treatment Decreases Survival of Multiple Cancer Cell Types

Different combinations of oligonucleotides were annealed and transfected into onco-SNP (LCS6 SNP) positive cell lines IGR-OV1 (ovarian), DU-145 (prostate), 789-0 (renal), MIAPACA (pancreatic), EKVX (lung) and MCF7 (breast) (see, Example 6, paragraph 176, for delineation of combinations). Cells were grown in clonogenic assays to determine the affect of the oligos on cell survival (see, Example 1 for clonogenic assay explanation).

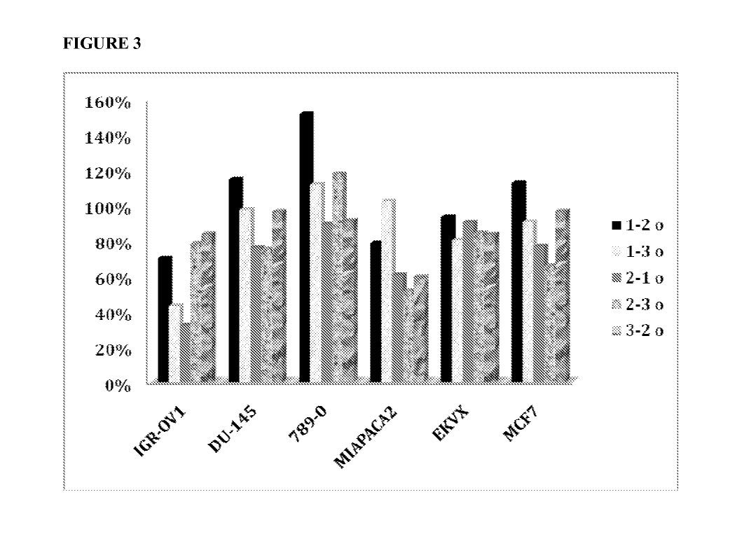

FIG. 3 shows that administration of one or more modified oligos leads to a decrease of cancer cell survival when that cancer cell contains the LCS6 SNP. The most dramatic results were observed with the ovarian, lung, breast, and pancreatic cancer cells lines. While more varied, effective cell death was observed in the prostate and renal cell lines when several of the oligonucleotide combinations were used. Thus, the data of FIG. 3 show that administration of the modified oligonucleotides of the invention are an effective broad-spectrum treatment for cancers containing the LCS6 SNP

The variation in efficacy between oligonucleotide combinations within a single tumor type can be explained in a number of ways. First, these oligonucleotide combinations contain different RNA to DNA ratios. As explained in Example 2, the use of RNA-DNA chimeras, as opposed to double-stranded RNA chimeras, can minimize off-targeting by a plurality of mechanisms, such as inhibiting introduction of the passenger strand (PS) into the RISC and rendering the PS non-functional during miRNA processing, as well as increasing the specificity of the seed sequence binding of the miRNA to the mRNA target (which lowers the binding energy between the miRNA and its mRNA making the interaction more favorable and more probable). Second, as opposed to the binding events demonstrated in FIG. 2B, in which the modified oligonucleotide targeted a synthetic and exogenously introduced construct containing the 3′UTR of KRAS fused to a luciferase gene, the oligonucleotides of FIG. 3 target an endogenous KRAS mRNA. This second difference is significant because endogenous mRNAs have secondary structure that affects the availability of miRNA binding sites and the binding energies required for miRNA-mRNA interactions. Moreover, mRNAs are expressed at physiological levels that vary between cell lines, even those originating from the same tissue. mRNAs are also targeted by a variety of endogenous regulatory RNAs and proteins that compete with the modified oligonucleotide.

The cell lines used in FIG. 3 were chosen not only because they contain the LCS6 SNP, but also because they are art-recognized models of these cancer types. However, the ordinarily skilled artisan would readily recognize that a cell line, particularly one which has been maintained in vitro for many generations, even though it contains the LCS6 SNP and overexpresses KRAS, may not be dependent upon KRAS for its oncogenic capabilities. Thus, even if the modified oligonucleotides effectively silenced KRAS in the cell lines of FIG. 3, the cell survival may not have been effected in all clones. Thus, the efficacy of modified oligonucleotides of the invention is verified in primary tumor cells.

To further explain the individual variation, the LCS6 SNP cancer cell lines may express varying levels of endogenous let-7 miRNAs or varying alleles of let-7 miRNAs that compete with the modified oligonucleotides for binding the LCS6 SNP site. Although it is expected that an endogenous let-7 miRNA should be less efficacious at binding to the LCS6 SNP, it is possible that the sequences of some modified oligonucleotides allow far more favorable binding interactions and enable these oligos to better out-compete endogenous and ineffective miRNAs. To overcome this competition, the dosage of the modified oligonucleotide is increased. Alternatively, or in addition, more than one modified oligonucleotide is administered simultaneous or sequentially to a subject until a therapeutically favorable result is achieved.

Finally, any given modified oligonucleotide could have a sequence that is substantially similar to the mRNA target of an endogenous miRNA, and therefore, could be targeted by the host cell for degradation. To overcome this occurrence, either another modified oligonucleotide having a different sequence that also binds the LCS6 SNP is simultaneously or sequentially administered to the subject.

Critically, the data of FIG. 3 are robust and demonstrate that the modified oligonucleotide combinations of the invention effectively decreased cell survival of each LCS6 SNP containing cancer cell type, overcoming each of the above obstacles, which are plausible and may explain the few outliers. Furthermore, because FIG. 3 demonstrates that in every cancer cell type containing the LCS6 SNP, at least one, and in fact, multiple modified oligonucleotides effectively decreased cancer cell survival. Thus, administration of multiple modified oligonucleotides should ensure a positive therapeutic result provided the data of FIG. 3.

The efficacy of administration of a composition containing a modified oligonucleotide to a cell is determined by comparing the efficacy of decreasing cancer cell survival of those cells, either LCS6-SNP positive or -negative, which received the modified oligo to those cells, either LCS6-SNP positive or -negative, which either received a negative oligonucleotide control oligo or a placebo negative control. An exemplary negative oligonucleotide control oligo is the Allstars negative control oligo, which is an annealed double stranded siRNA, available from Qiagen.

### Example 8

### Modified Oligo Treatment Decreases Survival of Ovarian and Pancreatic Cancer Cell Types

Different combinations of oligonucleotides were annealed and transfected into onco-SNP (LCS6 SNP) positive ovarian and pancreatic cell lines (see, Example 6, paragraph 176, for delineation of combinations). Cells were grown in clonogenic assays to determine the affect of the oligos on cell survival (see, Example 1 for clonogenic assay explanation).

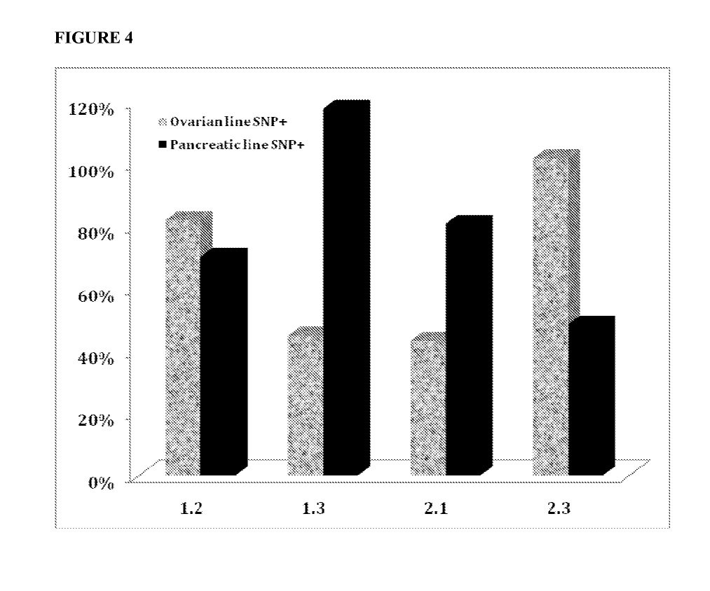

FIG. 4 shows that administration of one or more modified oligos leads to a decrease of cancer cell survival when that cancer cell contains the LCS6 SNP, compared to cancer cells that do not contain the LCS6 SNP. Similar to FIG. 3, and with very few exceptions, modified oligonucleotides of the invention effectively decreased cell survival of ovarian and pancreatic cancer cells when those cells contained the LCS6 SNP. Importantly, in each cancer type, FIG. 4 shows one oligonucleotide combination which decreased cancer cell survival by at least 50%. Ovarian cancer cell survival decreased by more 50% following the administration of oligonucleotide combinations 1.3 and 2.1. Similarly, pancreatic cancer cell survival decreased by at least 50% following the administration of oligonucleotide combination 2.3.

### Example 9

### Modified Oligo Treatment Decreases Survival of Ovarian Cancer Cells

Different combinations of oligonucleotides were annealed and transfected into an onco-SNP (LCS6 SNP) positive ovarian cell line and an onco-SNP negative cell line (see, Example 6, paragraph 176, for delineation of combinations). Cells were grown in clonogenic assays to determine the affect of the oligos on cell survival (see, Example 1 for clonogenic assay explanation).

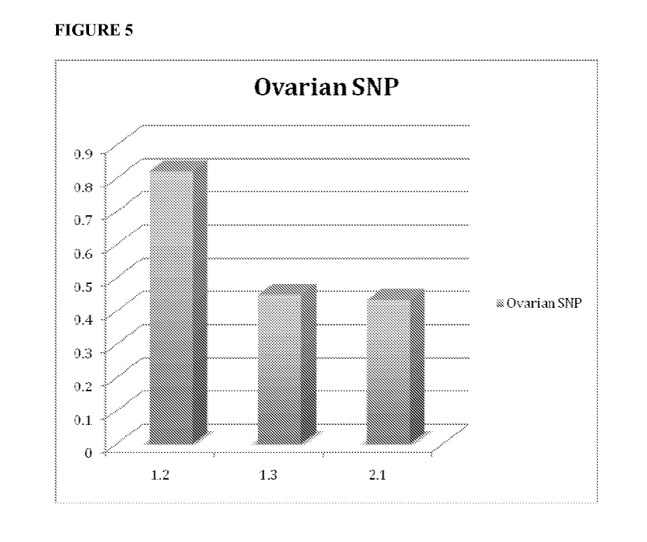

FIG. 5 shows that administration of one or more modified oligonucleotides leads to decreased cancer cell survival when that cancer cell contained the LCS6 SNP, compared to cancer cells that did not contain the LCS6 SNP. All of the modified oligonucleotide combinations shown effectively decreased LCS6 SNP positive cancer cell survival compared to the SNP-negative ovarian cancer cell line. Similar to FIG. 4, modified oligonucleotide combinations 1.3 and 2.1 decreased ovarian cancer cell survival by more than 50%.

### Example 10

### Computational Prediction of Modified Oligonucleotide Binding

To determine whether modified oligonucleotides of the invention bind the LCS6 SNP, or another oncogene, and what amount of binding energy is required for the interaction to occur, a computational approach is used. For instance, a RNAhybrid, a publicly-available algorithm, is used to compare the binding energy of a modified oligonucleotide of the invention and a known miRNA to a given target RNA (see, Kruger, J. and Rehmsmeier, M. Nucleic Acids Research, 2006, 34:W451-454; Rehmsmeier, M. and Steffen, P. RNA, 2004, 10:1507-1517). One advantage of the RNAhybrid program is that parameters such as the length of the seed sequence, G:U base pairing, and a seed-match option can be modified to account for non-canonical interactions.

Thus, to use the above algorithm for the treatment of a subject, one or more KRAS alleles of a cancer subject is amplified (for instance, using polymerase chain reaction, PCR), particularly within the 3′UTR, and this portion of the amplified gene is sequenced according to methods known in the art. Once the DNA sequence of the KRAS gene is determined, the mRNA sequence is delineated according to the following rules: adenosine (DNA) pairs with uracil (RNA), thymine (DNA) pairs with adenosine (RNA), cytosine (DNA) pairs with guanine (RNA), and guanine (DNA) pairs with cytosine (RNA). The mRNA sequence of the KRAS transcript is compared to the sequence of a modified oligonucleotide of the invention using RNAhybrid. If the binding energy required for the modified oligonucleotide to bind to the KRAS gene is less than the binding energy for a wild type or endogenous let-7 miRNA to bind to the same KRAS gene (this interaction is used as a control), then the modified oligonucleotide therapeutically silences KRAS. It is expected when a modified oligonucleotide has a more favorable binding energy than a wild type let-7 miRNA, the KRAS gene contains a mutation. That mutation is most often the LCS6 SNP.

### Example 11

### Experimental Validation of Modified Oligonucleotide Efficacy in Primary Tumor Cells

In order to determine the effectiveness of a modified oligonucleotide of the invention in treating the cancer or tumor of a subject, a biopsy is performed, and primary tumor cells are removed. These ex vivo primary tumor cells, either taken from the same patient for which treatment is intended, or from patient of similar medical background, are tested for the presence or absence of the LCS6 SNP. Although it is expected that treatment with a modified oligonucleotide of the invention is more efficacious in those subjects who carry the LCS6 SNP mutation, compositions of the invention are intended for all KRAS- or RAS-dependent tumors. The term “similar medical background” is meant to describe a subject of equivalent age, gender, height, weight, medical history (e.g. history of medical conditions), LCS6 SNP status, cancer type and stage, and treatment-regime.

Alternatively, or in addition, combinations of modified oligonucleotides are annealed and transfected into primary tumor cells isolated from leukemia, lymphoma, carcinoma, sarcoma, germ cell cancers, or blastoma cancers (see, Example 6, paragraph 176, for exemplary combinations). Cells are grown in clonogenic assays to determine the affect of the modified oligos on cell survival (see, Example 1 for clonogenic assay explanation). Treated primary cancer cells are compared to untreated LCS6-SNP positive, treated LSC6-SNP negative, and/or untreated LCS6-SNP negative primary cancer cells.

Subjects whose primary cancer cells are responsive to modified oligonucleotides of the invention, e.g. primary cancer cells show decreased cell survival when compared to non-SNP containing primary cancer cells or to primary cells that are not treated with a composition containing a modified oligonucleotide of the invention, are administered a composition including at least one modified oligonucleotide of the invention, provided locally or systemically. Most often the composition administered to the subject contains the same modified oligonucleotide administered to the primary cancer cells. Optionally, a composition containing more than one modified oligonucleotide is used in the primary cancer cell test as well as the treatment of the subject.

### Example 12

### Experimental Validation of Modified Oligonucleotide Efficacy and Delivery in Mouse Xenograft Models

An established human cancer cell line, as described in Example 7, or primary human cancer cells, as described in Example 11, is introduced into an immune-compromised mouse tumor model to determine the efficacy of treatment and delivery of modified oligonucleotide compositions in vivo. For instance, a mouse with decreased numbers of B-, T-, or Natural Killer (NK)-cells compared to a wild type mouse is considered immunocompromised. Exemplary immunocompromised mice commonly used for xenograft experiments include, but are not limited to, SCID, NOD, NSG (NOD SCID gamma), and Nude athymic mice. Additional examples can be found, for instance, at Jackson Laboratories' listing of mouse strains used as research tools for cancer (http://jaxmice.jax.org/list/rax3.html).

Xenograft mouse models of the invention are implanted with cancer cell lines or primary cancer cells which, optionally, carry the LCS6 SNP mutation and respond to modified oligonucleotide treatment in vitro. Cells are implanted either subcutaneously or orthotopically, e.g. at the site of the tumor origin to recapitulate the original human condition. Xenografts are either treated with a composition of the invention or a control treatment (for instance, an antisense or scrambled oligonucleotide) and evaluated for changes in a variety of parameters including, but not limited to, tumor grade, tumor size, tumor encapsulation, metastatic potential or metastasis, tumor regression, cancer remission, tumor vascularization, tumor cell-mediated secretion of angiogenic or growth factors, tumor cell-mediated degradation of cellular matrix surrounding the tumor, and cancer cell survival or death. Exemplary signs of therapeutic effectiveness include improvements in tumor grade; decreases in tumor size; maintenance of tumor encapsulation; maintenance or decrease of metastatic potential; prevention, inhibition, or suspension of metastasis; maintenance or increased in tumor regression; maintenance of cancer remission; prevention, inhibition, or reversal of tumor vascularization; prevention or inhibition of tumor cell-mediated secretion of angiogenic or growth factors; prevention or inhibition of tumor cell-mediated degradation of cellular matrix; decrease in cancer cell survival; increase in cancer cell death. Moreover, prolonged survival of the mouse is also a sign of therapeutic efficacy.

Mice containing xenographs are monitored for signs of toxicity following treatment. Dosages of compositions of the invention are increased until the maximal tolerable amount is determined. In other terms, the no observed adverse effect levels (NOAELs) are determined. These dosages levels are translated using the United States Federal Drug Administration's guidelines to determine the Human Equivalent Dosage as a maximal recommended starting dose (MRSD) for human clinical trials. This conversion is typically based upon body surface area when a composition of the invention is administered systemically. However, when a composition is administered by a route for which the dose is limited to local toxicity (e.g., topical, intranasal, subcutaneous, intramuscular) the human concentration is normalized to concentration or amount of composition at the application site. Moreover, when the composition is administered to an anatomical compartment with little subsequent systemic distribution (e.g. intrathecal, intravesical, intraocular, intrapleural, and intraperitoneal), the human concentration should be normalized to the compartmental volumes and concentrations of the composition.

In a preferred embodiment of the invention, the therapeutically effective dose of the composition is administered to the mouse with the xenograft or to a human subject and the concentration of the modified oligonucleotide in the blood or cerebral spinal fluid (CSF) of the individual is determined by calculating the area under the curve on a graph of dose over time. The optimal treatment regime of the individual can be determined by maintaining a constant blood or CSF concentration of a composition of the invention, even though the dosage required to reach that concentration would vary within an individual or between individuals.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated modified oligo, wherein said modified oligo comprises the nucleotide sequence of SEQ ID NO: 22, 24, 25, 26, 27, 29, 30, 31, 32, 34, 35, or 36.

2. A composition comprising the isolated modified oligo of claim 1.

3. The composition of claim 2, further comprising a cytotoxic compound.

4. The composition of claim 3, wherein said cytotoxic compound is a radioactive isotope or is a chemotherapeutic compound.

5. A method of treating or alleviating a symptom of cancer comprising administering to a subject in need thereof the composition of claim 2.

6. The method of claim 5, wherein said cancer is lung cancer, ovarian cancer, breast cancer, uterine cancer, head and neck cancer, pancreatic cancer, prostate cancer, renal cancer, or colon cancer.

7. The method of claim 5, wherein said composition is administered locally.

8. The method of claim 5, wherein said composition is administered systemically.

9. The method of claim 5, wherein said composition is administered topically, intravenously, intraocularly, subcutaneously, intraparitoneally, intramuscularly, intraspinally, or surgically.

10. A method of inducing cell death in a cancer cell comprising contacting the composition of claim 2 to said cancer cell.

11. The method of claim 10, wherein said cancer cell is in vivo.

12. The method of claim 10, wherein said cancer cell is in vitro.

13. The method of claim 10, wherein said cancer cell is ex vivo.

14. The method of claim 10, wherein said cancer cell is in situ.

15. The method of claim 10, wherein said cancer is lung cancer, ovarian cancer, breast cancer, uterine cancer, head and neck cancer, pancreatic cancer, prostate cancer, renal cancer, or colon cancer.

16. The method of claim 10, wherein said composition is administered locally.

17. The method of claim 10, wherein said composition is administered systemically.

18. The method of claim 10, wherein said composition is administered topically, intravenously, intraocularly, subcutaneously, intraparitoneally, intramuscularly, intraspinally, or surgically.

19. The method of claim 5, wherein said subject carries the LCS6 SNP.

20. The method of claim 10, wherein said cancer cell contains a mutation.

21. The method of claim 20, wherein said mutation is the LCS6 SNP.
